(19) United States
(12) Patent Application Publication
(10) Pub. No.: US 2025/0346696 A1
(43) Pub. Date: Nov. 13, 2025

(54) METHOD FOR PRODUCING PROPYLENE-BASED POLYMER

(71) Applicant: JAPAN POLYPROPYLENE CORPORATION, Tokyo (JP)

(72) Inventors: Tomohiro HOSOI, Tokyo (JP); Takahisa NISHIBU, Mie (JP)

(73) Assignee: JAPAN POLYPROPYLENE CORPORATION, Tokyo (JP)

(21) Appl. No.: 18/875,419

(22) PCT Filed: May 30, 2023
(86) PCT No.: PCT/JP2023/020011

§ 371 Date: Dec. 16, 2024

(30) Foreign Application Priority Data
Jun. 24, 2022 (JP) 2022-101618

(51) Int. Cl. C08F 210/06

(52) U.S. Cl. CPC C08F 210/06

(57) ABSTRACT

To provide a method for producing a propylene-based polymer, wherein, in a first step, in a presence of an olefin polymerization catalyst, a first propylene-based polymer is produced; in a subsequent second step, in a presence of the first propylene-based polymer, a second propylene-based polymer is produced; and wherein at least one selected from the group consisting of the first step, the second step and a midpoint of the first and second steps includes addition of a biomass-derived reaction inhibitor containing 5 ppm by mass to 2000 ppm by mass of water.

### TECHNICAL FIELD

The present invention relates to a method for producing a propylene-based polymer.

### BACKGROUND ART

A propylene-based polymer obtained by propylene homopolymerization or copolymerization of propylene and a comonomer in the presence of an olefin polymerization catalyst is lightweight, excellent in stiffness, heat resistance and chemical resistance, and low-cost among thermoplastic resins. Accordingly, the propylene-based polymer is widely used in automobile components such as an interior material and a bumper, and in many general household appliances.

In the production of such a propylene-based polymer, using multiple polymerization reactors, each of polymers having different molecular weights and comonomer contents is polymerized in each of the polymerization reactors, thereby imparting a wide distribution of composition to the propylene-based polymer and improving the function of the final product. For example, such a method is implemented, that after a low-molecular-weight polymer in is polymerized a previous polymerization reactor, a high-molecular-weight polymer is polymerized in a next polymerization reactor to extend the molecular weight distribution of the polymer, thereby improving the moldability thereof. Also, such a method is implemented, that a crystalline propylene homopolymer is polymerized in a previous polymerization reactor, and an amorphous propylene-ethylene copolymer having a large comonomer content is polymerized in a next polymerization reactor to produce a so-called propylene-based block copolymer, thereby improving the balance between the stiffness and impact resistance of the propylene-based polymer.

In such a propylene-based polymer, the content rate of each polymer produced in each polymerization reactor has a strong influence on the physical properties of the final product. Accordingly, it is known to add, in the production process of the propylene-based polymer, a reaction inhibitor having the function of deactivating the olefin polymerization catalyst. For example, Patent Documents 1 and 2 disclose the use of an active hydrogen compound (e.g., alcohol) as the reaction inhibitor, from the viewpoint of not only controlling the content rate of each of the propylene-based polymers, but also suppressing the adhesion of the propylene-based polymers and quality deterioration such as the resulting formation of aggregated lump polymers, gel, etc.

Meanwhile, in recent years, there is an attempt to reduce pressure on the environment or to create a recycling-based society by switching a petrochemical-derived raw material to a biomass-derived raw material. For example, Patent Document 3 proposes a wrapping film in which a resin film, which contains polyester obtained by use of biomass-derived ethylene glycol and fossil fuel-derived dicarboxylic acid, is used as the base layer. Patent Document 4 reports the development of a process in which propylene is finally produced from a biomass raw material and biopolypropylene is produced by use of the propylene.

Also, Non-Patent Document 1 metal-supported porous carbon on the removal of impurities that can inhibit polymerization activity, when implementing the production of polypropylene from a biomass raw material.

### CITATION LIST

### Patent Documents

Patent Document 1: Japanese Patent Application Laid-Open (JP-A) No. S61-69821
Patent Document 2: JP-A No. 2001-261720
Patent Document 3: JP-A No. 2021-91228
Patent Document 4: International Publication No. WO2007/055361

### Non-Patent Document

Non-Patent Document 1: “Liquid phase adsorption characteristics of metal-supported porous carbon for removal of sulfur-based impurities from bioethanol”, The Society of Chemical Engineers of Japan (SCEJ), Proceedings of SCEJ Annual Meeting, 2009, p. 216

### SUMMARY OF INVENTION

### Technical Problem

As described above, a propylene-based polymer is widely used as industrial sheets, automobile components and so on, due to having excellent properties. On the other hand, from the viewpoint of environmental protection, a propylene-based polymer is required to reduce the usage of a fossil resource-derived raw material as much as possible, which is used in its production process.

However, since a compound derived from a biomass raw material contains impurities and foreign substances, there are concerns that a decrease in the quality of the final product or interference with long-term continuous production may occur when used.

In light of the above-described problems with the prior art, an object of the present invention is to provide a propylene-based polymer production method in which, even when a biomass-derived reaction inhibitor containing impurities is used in the production, a propylene-based polymer can be produced without causing a remarkable decrease in productivity and a long-term unstable operation owing to the formation of lump polymers.

### Solution to Problem

As a result of an extensive study, the inventors of the present disclosure found that the impurities contained in a reaction inhibitor derived from a biomass raw material fall within a certain range, the reaction inhibitor is applicable to polypropylene polymerization, without causing problems such as a decrease in catalytic activity, the formation of lump polymers and a change in color and odor. Based on these findings, the inventors of the present disclosure at last achieved the present invention.

The present invention relates to the following propylene-based polymer production methods [1] to [7].

[1] A method for producing a propylene-based polymer,
wherein, in a first step, using one or two or more polymerization reactors and in a presence of an olefin polymerization catalyst, a propylene homopolymer or a copolymer of propylene and at least one kind of monomer selected from the group consisting of α-olefins containing 2 to 10 carbon atoms other than propylene, is produced as a first propylene-based polymer;
wherein, in a subsequent second step, using one or two or more polymerization reactors and in a presence of the first propylene-based polymer, a propylene homopolymer or a copolymer of propylene and at least one kind of monomer selected from the group consisting of α-olefins containing 2 to 10 carbon atoms other than propylene, is produced as a second propylene-based polymer; and
wherein at least one selected from the group consisting of the first step, the second step and a midpoint of the first and second steps includes addition of a biomass-derived reaction inhibitor containing 5 ppm by mass to 2000 ppm by mass of water.

[2] The method for producing the propylene-based polymer according to the above-described [1], wherein the reaction inhibitor further contains 0.1 ppm by mass to 1000 ppm by mass of methanol.

[3] The method for producing the propylene-based polymer according to the above-described [1] or [2], wherein the reaction inhibitor further contains 0.1 ppm by mass to 5 ppm by mass of sulfur atoms and 0.1 ppb by mass to 100 ppb by mass of copper atoms.

[4] The method for producing the propylene-based polymer according to any one of the above-described [1] to [3], wherein the reaction inhibitor is biomass-derived ethanol.

[5] The method for producing the propylene-based polymer according to any one of the above-described [1] to [4], wherein the olefin polymerization catalyst contains a solid catalyst component (A) containing the following (A1), (A2) and (A3) and optionally containing the following (A4), and the following component (B):
(A1) a solid component containing magnesium, titanium, a halogen and an electron donating compound serving as an internal donor
(A2) an organoaluminum compound
(A3) an organosilicon compound other than a vinylsilane compound
(A4) a vinylsilane compound
(B) an organoaluminum compound.

[6] The method for producing the propylene-based polymer according to any one of the above-described [1] to [5], wherein the second propylene-based polymer produced in the second step is a copolymer of propylene and at least one kind of monomer selected from the group consisting of α-olefins containing 2 to 10 carbon atoms other than propylene, and a content of the at least one kind of monomer selected from the group consisting of α-olefins containing 2 to 10 carbon atoms other than propylene, is in a range of from 20% by mass to 80% by mass.

[7] The method for producing the propylene-based polymer according to the above-described [5] or [6], wherein an amount of the added reaction inhibitor is from 0.01 g to 30 g with respect to 1 g of a total amount of the solid catalyst component (A).

### Advantageous Effects of Invention

According to the present invention, a continuous, multi-step propylene-based polymer production method can be provided, which is configured to produce a propylene-based polymer without causing an excessive decrease in catalytic polymerization activity and unstable continuous production owing to the formation of lump polymers, even when a reaction inhibitor derived from a biomass raw material is used.

### DESCRIPTION OF EMBODIMENTS

The propylene-based polymer production method of the present invention is a method for producing a propylene-based polymer,
wherein, in a first step, using one or two or more polymerization reactors and in a presence of an olefin polymerization catalyst, a propylene homopolymer or a copolymer of propylene and at least one kind of monomer selected from the group consisting of α-olefins containing 2 to 10 carbon atoms other than propylene, is produced as a first propylene-based polymer;
wherein, in a subsequent second step, using one or two or more polymerization reactors and in a presence of the first propylene-based polymer, a propylene homopolymer or a copolymer of propylene and at least one kind of monomer selected from the group consisting of α-olefins containing 2 to 10 carbon atoms other than propylene, is produced as a second propylene-based polymer; and
wherein at least one selected from the group consisting of the first step, the second step and a midpoint of the first and second steps includes addition of a biomass-derived reaction inhibitor containing 5 ppm by mass to 2000 ppm by mass of water.

In the present invention, the biomass-derived reaction inhibitor containing 5 ppm by mass to 2000 ppm by mass of water is added in at least one selected from the group consisting of the first step, the second step and the midpoint of the first and second steps. Accordingly, with suppressing an excessive decrease in polymerization catalytic activity and without causing a long-term unstable operation owing to the formation of lump polymers, the desired propylene-based polymer can be produced while reducing pressure on the environment. By using, as an alternative substance to a fossil resource-derived reaction inhibitor, the reaction inhibitor derived from the biomass raw material containing an impurity at a specific concentration or less, the desired propylene-based polymer can be produced while reducing pressure on the environment.

Hereinafter, the embodiment of the present invention will be described in detail. The descriptions of the components described below are merely examples of the embodiments of the present invention, and the present invention is not limited to the contents of the following description, unless it is beyond the gist thereof.

In the present Description, “to” which shows a numerical range is used to describe a range in which the numerical values described before and after “to” indicate the lower limit value and the upper limit value.

### I. Method for Producing Propylene-Based Polymer

### 1. Polymerization Step

In the production method of the present invention in which, in the first step, the first propylene-based polymer is polymerized using one or two or more polymerization reactors and in the presence of the below-described olefin polymerization catalyst, and in the subsequent second step, the second propylene-based polymer is polymerized using one or two or more polymerization reactors and in the presence of the first propylene-based polymer, the biomass-derived reaction inhibitor containing at least 5 ppm by mass to 2000 ppm by mass of water as an impurity, is added in at least one selected from the group consisting of the first step, the second step and the midpoint of the first and second steps.

The polymerization style of the production method of the present invention can use any commonly known method such as bulk polymerization, vapor phase polymerization, solution polymerization and slurry polymerization, as long as the olefin polymerization catalyst is efficiently brought into contact with a monomer. From the viewpoint of economy, the most preferred is the vapor phase polymerization method in which monomers are kept in gaseous form without the substantial use of a liquid solvent so that production efficiency per catalyst can be improved. As the polymerization method, a continuous or batch polymerization method is used.

One or more polymerization reactors (e.g., two or more) may be used in both the first and second steps. The first step is carried out in one or two or more vapor phase polymerization reactors, and the second step is carried out in one or two or more vapor phase polymerization reactors. When several polymerization reactors are used, they may be connected in series or in parallel.

As the vapor phase polymerization reactor, examples include, but are not limited to, a fluid bed reactor and a horizontal reactor equipped with an agitator inside, which rotates around a horizontal axis thereof.

As the monomer used for polymerization in the first or second step, in the case of producing the propylene homopolymer, a single propylene monomer is used.

In the case of producing the copolymer of propylene and at least one kind of monomer selected from the group consisting of α-olefins containing 2 to 10 carbon atoms other than propylene, a monomer mixture such that at least one kind of comonomer selected from the group consisting of ethylene and α-olefins containing 4 to 10 carbon atoms is incorporated in propylene, is used as a monomer and as a raw material in the first or second step. As the α-olefins containing 4 to 10 carbon atoms, examples include, but are not limited to, 1-butene, 1-pentene, 4-methyl-1-pentene, 3-methyl-1-pentene, 1-hexene, 1-heptene, 1-octene, 1-nonene and 1-decene.

The polymerization temperature is preferably from 0° C. to 90° C., more preferably from 30° C. to 85° C., and still more preferably from 45° C. to 80° C. The polymerization pressure is preferably from 0.1 MPaG to 5 MPaG, and more preferably from 0.5 MPaG to 4 MPaG.

In general, by selecting a higher temperature and a higher pressure, productivity per gram of a catalyst can be increased; however, on the other hand, local heat cannot be removed, and fine powder is produced by the collapse of grown particles, or an aggregate or lump is formed by fusion. Accordingly, in consideration of balance between the productivity per gram of the catalyst and the removal of local heat, the polymerization temperature and the polymerization pressure are adjusted in the above temperature range and pressure range, respectively.

The residence time can be freely adjusted according to the structure of a polymerization reactor. In general, it is set within a range of from 30 minutes to 10 hours. The residence time is preferably 4 hours or less, and more preferably 3 hours or less. In general, by selecting a longer residence time, productivity per gram of a catalyst can be increased. However, when the residence time is too long, a productivity increase rate per gram of a catalyst with respect to an increase in the residence time decreases. Accordingly, considering the productivity per gram of a catalyst, the residence time is adjusted in the above range.

In the propylene-based polymer production method of the present invention, from the viewpoint of productivity, 10000 g or more of the first propylene-based polymer is preferably produced in the first step per gram of the below-described olefin polymerization catalyst.

### 2. Biomass-Derived Reaction Inhibitor

As the biomass-derived reaction inhibitor in the present invention, at least one of an alcohol compound and an ethylene glycol-containing compound can be used. From the viewpoint of reducing pressure on the environment, the reaction inhibitor is preferably an agent that can be synthesized from a biomass raw material, particularly preferably an agent that can be synthesized from a plant-derived raw material. This is because plants absorb and consume carbon dioxide by photosynthesis in the process of growth. Among biomass-derived reaction inhibitors, more preferred is an alcohol compound or ethylene glycol-containing compound that can be synthesized using, as a starting material, bioethylene produced from plants.

From the viewpoint of relatively high safety on the human body and ease of handling during production, an alcohol compound is most preferred.

In the present invention, whether or not the reaction inhibitor is derived from biomass can be determined by a commonly-known, bio-based content measurement method using isotopes such as ¹⁴C and ¹⁸O (e.g., ASTM D6866, a carbon isotope ¹⁴C ratio measurement method). The reaction inhibitor can be judged as a biomass-derived agent when the isotope exists. For example, to differentiate biomass-derived ethanol from petrochemical-derived ethanol, a method for measuring the content of a hydrogen isotope D or an oxygen isotope ¹⁸O by use of isotope ratio mass spectrometry or the like, can be used.

### 2-1. Biomass-Derived Alcohol Compound

The biomass-derived alcohol compound used in the reaction inhibitor of the present invention may be, for example, a compound represented by the following general formula (1).

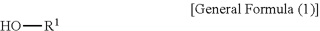

(where R¹ represents a saturated hydrocarbon group containing 2 to 10 carbon atoms.)

In the alcohol compound represented by the general formula (1), from the viewpoint of dry removal, a saturated hydrocarbon group containing 2 to 10 carbon atoms can be selected as a preferred example of R¹. R¹ is more preferably a saturated hydrocarbon group containing 2 to 8 carbon atoms, and still more preferably a saturated hydrocarbon group containing 2 to 3 carbon atoms.

When the number of the carbons of R¹ is more than 10, there is a high possibility that dry removal become difficult and the final product has a problem such as odor.

The biomass-derived alcohol compound is most preferably ethanol containing 2 carbon atoms (bioethanol), which is widely produced deriving from a biomass raw material.

### 2-2. Biomass-Derived, Ethylene Glycol-Containing Compound

The biomass-derived, ethylene glycol-containing compound used in the reaction inhibitor of the present invention may be, for example, a compound represented by the following general formula (2) or (3).

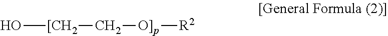

(where p is an integer and satisfies 1≤p≤10, and R² represents a hydrogen atom or a hydrocarbon group containing 1 to 25 carbon atoms.)

Since the polyoxyethylene skeleton has high hydrophilicity, there may be limitations on handling during production, such as a decrease in solubility in organic solvents and solidification at ordinary temperature, when p is increased. Accordingly, 1≤p≤10 can be preferably selected.

More specifically, as the biomass-derived, ethylene glycol-containing compound, examples include, but are not limited to, ethylene glycol, diethylene glycol, polyoxyethylene(3)lauryl ether, polyoxyethylene(4)lauryl ether, polyoxyethylene(5)lauryl ether, polyoxyethylene(3)stearyl ether, ether, polyoxyethylene(4)stearyl polyoxyethylene(5)stearyl ether, polyoxyethylene(4)oleyl ether, and polyoxyethylene(6)oleyl ether. The number in the parentheses represents the polymerization degree of the polyoxyalkylene.

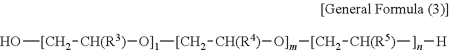

(where l, m and n are integers and satisfy all the following relations: 0≤l≤70, 0≤m≤70, 0≤n≤70 and 2≤l+m+n; R³, R⁴ and R⁵ each independently represent a hydrogen atom or a hydrocarbon group containing 1 to 5 carbon atoms; and a hydrogen atom is selected as at least one of R³, R⁴ and R⁵, and when several hydrocarbon groups are selected, they may be identical to or different from each other.)

When R³, R⁴ and R⁵ are each independently a hydrogen atom, that is, when they are each independently polyoxyethylene, since the polyoxyethylene skeleton has high hydrophilicity and has crystallinity, an increase in the abundance thereof may cause limitations on handling during production, such as a decrease in solubility in organic solvents and solidification at ordinary temperature. Accordingly, 70 can be preferably selected as the upper limit value of the polymerization degree of the polyoxyethylene, that is, the upper limit values of l, m and n.

When R³, R⁴ and R⁵ are each independently a hydrocarbon group, there is a possibility that the molecular weight increases according to the number of the carbon atoms, resulting in an increase in viscosity and a decrease in affinity for solvents and imposing limitations on handling. Accordingly, 5 can be preferably selected as the upper limit of the number of the carbon atoms. When R³, R⁴ and R⁵ are each independently a hydrocarbon group, most preferred is a propylene oxide where R³, R⁴ and R⁵ each contain one carbon atoms.

As such compounds, examples include, but are not limited to, polyoxyethylene(1)polyoxypropylene(16)polyoxyethylene(1), polyoxyethylene(2)polyoxypropylene(16)polyoxyethylene(2), polyoxyethylene(2)polyoxypropylene(30)polyoxyethylene(2), polyoxyethylene(6)polyoxypropylene(35)polyoxyethylene(6), polyoxyethylene(5)polyoxypropylene(69)polyoxyethylene(5), polyoxyethylene(3)polyoxypropylene(2)polyoxyethylene(3)lauryl ether, polyoxyethylene(3)polyoxypropylene(3)lauryl ether, polyoxypropylene(26)polyoxyethylene(6), polyoxypropylene(26), polyoxypropylene(26)polyoxyethylene(7), polyoxypropylene(26), polyoxypropylene(22)polyoxyethylene(6), polyoxypropylene(22), polyoxypropylene(12)polyoxyethylene(14) and polyoxypropylene(12). The number in the parentheses represents the polymerization degree of the polyoxyalkylene.

The ethylene glycol-containing compound used in the biomass-derived reaction inhibitor of the present invention, may be a single component or a mixture of components.

Also, the biomass-derived reaction inhibitor may be one kind of reaction inhibitor, may be a mixture of two or more kinds of reaction inhibitors, or may be a mixture of the alcohol compound and the ethylene glycol-containing compound.

### 3. Impurities in the Biomass-Derived Reaction Inhibitor

As the impurities contained in the biomass-derived reaction inhibitor of the present invention, examples include, but are not limited to, water, methanol, a sulfur compound and a copper compound.

For example, in the case of alcohols produced by fermentation from biological resources, such as bioethanol, as the impurities, examples include, but are not limited to, water, methanol, a sulfur compound such as dimethyl sulfide, and a copper compound such as copper acetate, which can be mixed during the production process.

The content of the water in the biomass-derived reaction inhibitor is preferably in a range of from 5 ppm by mass to 2000 ppm by mass. The lower limit value may be 20 ppm by mass or more, may be more than 100 ppm by mass, may be 200 ppm by mass or more, or may be 500 ppm by mass or more. The upper limit value may be 1800 ppm by mass or less, or it may be 1700 ppm by mass or less.

In the case of using a mixture of two or more kinds of reaction inhibitors, the total water content in the reaction inhibitors is adjusted in the above-mentioned range such as a range of from 5 ppm by mass to 2000 ppm by mass.

When the water content is above the range, there is a production problem such that the water deposits in a production equipment and induces failure of the equipment.

When the water content is below the range, it means that according to the refinement degree thereof, energy for impurity removal is used. Accordingly, even when the reaction inhibitor is derived from biomass, an advantage in the pressure on the environment weakens.

The content of the methanol as an impurity in the biomass-derived reaction inhibitor is preferably in a range of from 0.1 ppm by mass to 1000 ppm by mass, from the viewpoints of ensuring safety on the human body when handing in the propylene-based polymer production process, and suppressing odor and improving product safety by reducing the remaining amount in the final product, and increasing the safety of the product. As for the content of the methanol as an impurity in the biomass-derived reaction inhibitor, the lower limit may be 30 ppm by mass or more, may be 100 ppm by mass or more, may be more than 200 ppm by mass, or may be 300 ppm by mass or more. The upper limit may be 700 ppm by mass or less, or it may be 500 ppm by mass or less.

The content of the sulfur atoms as an impurity in the biomass-derived reaction inhibitor is preferably in a range of from 0.1 ppm by mass to 5 ppm by mass, from the viewpoint of suppressing a change in the quality of the product, such as odor and color.

As for the content of the sulfur atom as an impurity in the biomass-derived reaction inhibitor, the lower limit may be 0.2 ppm by mass or more, or it may be 0.3 ppm by mass or more. The upper limit may be 4 ppm by mass or less, or it may be 3 ppm by mass or less.

When the sulfur atoms content is below the range, according to the refinement degree thereof, energy for impurity removal is needed. Accordingly, even when the reaction inhibitor is derived from a biomass raw material, an advantage in the pressure on the environment weakens.

The content of the copper atoms as an impurity in the biomass-derived reaction inhibitor is preferably in a range of from 0.1 ppb by mass to 100 ppb by mass, from the viewpoint of reducing the possibility of a decrease in quality which is due to a by-product unexpectedly mixed in the product. This is because an oxidation reaction that uses a copper ion as an active site, can occur as a side reaction.

As for the content of the copper atom as an impurity in the biomass-derived reaction inhibitor, the lower limit may be 1 ppm by mass or more, or it may be 10 ppm by mass or more. The upper limit may be 70 ppm by mass or less, or it may be 60 ppm by mass or less.

When the copper atoms content is below the range, according to the refinement degree thereof, energy for impurity removal is needed. Accordingly, even when the reaction inhibitor is derived from a biomass raw material, an advantage in the pressure on the environment weakens.

The biomass-derived reaction inhibitor used in the present invention is preferably such a reaction inhibitor, that when filtered through a filter paper No. 5C defined in JIS P 3801, nothing remains on the paper.

As the biomass-derived reaction inhibitor used in the present invention, at least one kind can be appropriately selected from commercially-available, biomass-derived alcohol compounds and ethylene glycol-containing compounds.

If the content of the impurities contained in the at least one kind selected from commercially-available, biomass-derived alcohol compounds and ethylene glycol-containing compounds, exceeds the content of the impurities specified in the present invention, before using the selected compound, the selected compound may be refined so that the content of the impurities in the compound is equal to or less than the specified content. A biomass-derived reaction inhibitor having a water content of more than 2000 ppm by mass may be passed through a column filled with, for example, molecular sieve 3 A or 4 A (available from Resonac Corporation, for example) to decrease the water content to a range of from 5 ppm by mass to 2000 ppm by mass, and then the reaction inhibitor may be used as the biomass-derived reaction inhibitor used in the present invention.

### 4. Supply of the Reaction Inhibitor

The reaction inhibitor is supplied in at least one selected from the group consisting of the first step, the second step and the midpoint of the first and second steps. From the viewpoint of suppressing a remarkable decrease in catalytic activity, the reaction inhibitor is preferably supplied at least at the midpoint of the first and second steps.

The method for supplying the reaction inhibitor in at least one selected from the group consisting of the first step, the second step and the midpoint of the first and second steps, is preferably any one of the following supplying methods.

(4-1) A method in which only one kind of compound is used as the reaction inhibitor and solely supplied to the polymerization reactor.

(4-2) A method in which two or more kinds of compounds are used as the reaction inhibitor and separately supplied to the polymerization reactor from different supply lines.

(4-3) A method in which several compounds are used as the reaction inhibitor; an alcohol compound and a polyethylene glycol-containing compound are mixed in advance; the mixture is supplied as a mixed reaction inhibitor to the polymerization reactor through a supply line; and oxygen is optionally supplied to the polymerization reactor from a different supply line.

(4-4) A method in which several compounds are used as the reaction inhibitor; an alcohol compound and a polyethylene glycol-containing compound are supplied from different supply lines and mixed in the supply lines; the mixture is fed to the polymerization reactor; and oxygen is optionally supplied to the polymerization reactor from a different supply line.

An ethylene glycol-containing compound has generally high viscosity. Accordingly, supplying an ethylene glycol-containing compound solely is economically disadvantageous in that more energy is required due to pressure loss in piping. Accordingly, in the case of using an ethylene glycol-containing compound, the method (4-3) or (4-4) is preferred.

However, this does not apply if an ethylene glycol-containing compound is not used as the reaction inhibitor. Either (4-2), in which the amount of each supply can be independently controlled, or (4-1) is the preferred method.

The position of the polymerization reactor used to supply the reaction inhibitor may be any position when the vapor phase polymerization reactor is a stirred tank reactor, and the position is preferably on the upstream side when the polymerization reactor is a plug flow reactor.

The amount of the supplied reaction inhibitor may be any amount so that the production rate of each polymerization step becomes a desired value. When the reaction inhibitor is supplied to the first step, the reaction inhibitor is preferably supplied so that the total amount of the supplied reaction inhibitor is in a range of from 0.01 g to 10 g per gram of the solid catalyst component (A) described below. The total amount is more preferably 0.5 g or more, still more preferably 1 g or more, and most preferably 3 g or more. The total amount may be 8 g or less, or it may be 5 g or less.

When the reaction inhibitor is supplied to the second step, similar to the above, the amount of the supplied reaction inhibitor may be any amount so that the production rate of each polymerization step becomes a desired value. The reaction inhibitor is preferably supplied so that the total amount of the supplied reaction inhibitor is in a range of from 10% by mass to 90000% by mass, and it is more preferably supplied so that the total amount of the supplied reaction inhibitor is in a range of from 2000% by mass to 85000% by mass, with respect to titanium in the solid catalyst component (A) supplied to the first step.

When the reaction inhibitor is supplied at the midpoint of the first and second steps, similar to the above, the amount of the supplied reaction inhibitor may be any amount so that the production rate of each polymerization step becomes a desired value. The reaction inhibitor is preferably supplied so that the total amount of the supplied reaction inhibitor is in a range of from 10% by mass to 90000% by mass, and it is more preferably supplied so that the total amount of the supplied reaction inhibitor is in a range of from 2000% by mass to 85000% by mass, with respect to titanium in the solid catalyst component (A) supplied to the first step.

Also, it is preferable to add the reaction inhibitor so that the total amount of the added reaction inhibitor is in a range of from 0.01 g to 30 g with respect to 1 g of the total amount of the solid catalyst component (A). With respect to 1 g of the total amount of the solid catalyst component (A) described below, the total amount of the added reaction inhibitor is more preferably 0.5 g or more, still more preferably 1 g or more, and most preferably 3 g or more. The total amount may be 20 g or less, or it may be 10 g or less. The “1 g of the total amount of the solid catalyst component (A)” does not include a preliminarily polymerized polymer described later.

When the total amount of the reaction inhibitor satisfies the above amount, the surface of the propylene-based polymer particles can be appropriately deactivated. Accordingly, the stickiness of the particles, which is widely caused by producing a component having a high comonomer content, can be suppressed, and there is an advantage such that the formation of lump polymers, which is due to the stickiness of the particles, can be suppressed, and the contamination of the wall surface of the reactor by the particles attached to the wall surface can be prevented.

The present invention does not restrict the use of an reaction inhibitor other than the biomass-derived reaction inhibitor. To the extent that does not significantly hinder the effects of the present invention, the reaction inhibitor other than the biomass-derived reaction inhibitor may be further contained. As the reaction inhibitor other than the biomass-derived reaction inhibitor, examples include, but are not limited a to, petrochemical-derived reaction inhibitor. However, from the viewpoint of increasing the effects of the present invention, with respect to the total amount of the reaction inhibitor used in the present invention, the biomass-derived reaction inhibitor may be 50% by mass or more, may be 70% by mass or more, may be 90% by mass or more, or may be 100% by mass.

### II. Olefin Polymerization Catalyst

The olefin polymerization catalyst used in the present invention is preferably a so-called Ziegler catalyst that contains components (A) and (B) as constitutional components, the component (A) being a solid catalyst component containing magnesium, titanium, a halogen and an electron donating compound serving as an internal donor, and the component (B) being an organoaluminum compound.

From the viewpoint of suppressing an excessive decrease in catalytic activity which is caused by the reaction inhibitor, the olefin polymerization catalyst preferably contains the solid catalyst component (A) containing the following (A1), (A2) and (A3) and optionally containing the following (A4), and the following component (B):
(A1) a solid component containing magnesium, titanium, a halogen and an electron donating compound serving as an internal donor
(A2) an organoaluminum compound
(A3) an organosilicon compound other than a vinylsilane compound
(A4) a vinylsilane compound
(B) an organoaluminum compound.

### 1. Component (A): Solid Catalyst Component

As the solid catalyst component containing magnesium, titanium, a halogen and an electron donating compound serving as an internal donor, a conventionally-known solid catalyst component can be used. In addition to the four components, the solid catalyst component may contain any component in any form, to the extent that does not impair the effects of the present invention.

### (A1a: Magnesium Source)

As the magnesium source of the solid catalyst component, any magnesium compound can be used. A typical example of the magnesium compound is a compound disclosed in JP-A No. H03-234707.

In general, the following can be used as the magnesium source: a halogenated magnesium compound as typified by magnesium chloride, an alkoxymagnesium compound as typified by diethoxymagnesium, magnesium metal, an oxymagnesium compound as typified by magnesium oxide, a hydroxymagnesium compound as typified by magnesium hydroxide, a Grignard compound as typified by butylmagnesium chloride, an organomagnesium compound as typified by butylethylmagnesium, a magnesium salt compound of an inorganic or organic acid as typified by magnesium carbonate and magnesium stearate, a mixture thereof, and a compound in which the average compositional formula is a formula formed by mixing them (e.g., a compound represented by Mg(OEt)ₘCl₂₋ₘ where 0<m<2).

Of them, preferred are magnesium chloride, diethoxymagnesium, magnesium metal and butylmagnesium chloride.

### (A1b: Titanium Source)

As the titanium source of the solid catalyst component, any titanium compound can be used. A typical example of the titanium compound is a compound disclosed in JP-A No. H03-234707. As for the valence of titanium, a titanium compound having any valence of 4, 3, 2 or 0 can be used. Preferred is a titanium compound having a valence of 4 or 3, and more preferably a titanium compound having a valence of 4.

As the titanium compound having a valence of 4, examples include, but are not limited to, a halogenated titanium compound as typified by titanium tetrachloride, an alkoxytitanium compound as typified by tetrabutoxytitanium, a condensation compound of alkoxytitanium having a Ti—O—Ti bond, as typified by tetrabutoxytitanium dimer (BuO)₃Ti—O—Ti(OBu)₃, and an organometallic titanium compound as typified by dicyclopentadienyl titanium dichloride. Of them, titanium tetrachloride and tetrabutoxytitanium are particularly preferred.

As the titanium compound having a valence of 3, examples include, but are not limited to, a halogenated titanium compound as typified by titanium trichloride. As the titanium trichloride, a compound produced by any known method can be used, such as a hydrogen reduction type compound, an aluminum metal reduction type compound, a titanium metal reduction type compound and an organoaluminum reduction type compound.

The above-mentioned titanium compounds can be used alone or in combination of two or more kinds. Also, a mixture of the above-mentioned titanium compounds, a compound in which the average compositional formula is a formula formed by mixing them (e.g., a compound represented by Ti(OBu)ₘCl₄₋ₘ where 0<m<4) or a complex with another compound such as phthalic acid ester (e.g., a compound represented by Ph(CO₂Bu)₂·TiCl₄) can be used.

### (A1c: Halogen)

The halogen in the solid catalyst component may be fluorine, chlorine, bromine, iodine or a mixture thereof. Of them, chlorine is preferred.

As the halogen source of the solid catalyst component, any of the above-mentioned halogenated magnesium and titanium compounds is commonly used. Besides them, another halogen source can be used, such as a known halogen compound such as a halogenated compound of aluminum (e.g., AlCl₃, AlBr₃, AlI₃), a halogenated compound of boron (e.g., BCl₃, BBr₃, BI₃), a halogenated compound of silicon (e.g., SiCl₄), a halogenated compound of phosphorus (e.g., PCl₃, PCl₅), a halogenated compound of tungsten (e.g., WCl₆) and a halogenated compound of molybdenum (e.g., MoCl₅).

### (A1d: Electron Donating Compound Serving as Internal Donor)

In the polymerization technique using a Ziegler catalyst, it is generally considered that the internal donor and the external donor have a different function.

The internal donor is a doner which is simultaneously used when the titanium compound is supported on the magnesium compound to form an active site. It controls the position to which a titanium atom is coordinated and changes the electronic state of the coordinated titanium atom.

Meanwhile, the external donor changes the nature of an already-formed active site. For example, by further using the external donor for the prepared solid catalyst component, the already-formed active site can be changed into a highly stereospecific active site, and an active site that can produce an amorphous component can be poisoned. Accordingly, a propylene-based polymer which has higher stereoirregularity and in which the content of the amorphous component is less, can be produced.

As the electron donating compound (internal donor), examples include, but are not limited to, an oxygen-containing electron donating compound such as an alcohol, a phenol, a ketone, an aldehyde, a carboxylic acid, an ester of an organic or inorganic acid, an ether, an acid amide and an acid anhydride, a nitrogen-containing electron donating compound such as ammonia, amine, nitrile and isocyanate, and a sulfur-containing electron donating compound such as sulfonic acid ester. In particular, examples include, but are not limited to, compounds mentioned in Paragraph 0037 of JP-A No. 2010-70584.

Of them, preferred are the following compounds: a phthalic acid ester compound as typified by diethyl phthalate, di-n-butyl phthalate, diisobutyl phthalate and diheptyl phthalate; a phthalic acid halide compound as typified by phthaloyl dichloride; an ester malonate compound having one or two substituents at the 2-position (e.g., 2-n-butyl-diethyl malonate); a succinic acid ester compound having one or two substituents at the 2-position or one or more substituents at the 2-position and the 3-position (e.g., 2-n-butyl-diethyl succinate); an aliphatic polyvalent ether compound having one or two substituents at the 2-position, as typified by 1,3-dimethoxypropane (e.g., 2-isopropyl-2-isobutyl-1,3-dimethoxypropane, 2-isopropyl-2-isopentyl-1,3-dimethoxypropane); and a polyvalent ether compound containing an aromatic free radical in the molecule, as typified by 9,9-bis(methoxymethyl)fluorene, for example.

These electron donating compounds can be used alone or in combination of two or more kinds.

The solid catalyst component of the present invention can be prepared by bringing the magnesium compound, the titanium compound, the halogen compound and the electron donating compound serving as the internal donor into contact with each other to form the solid component (A1) containing magnesium, titanium, a halogen and the electron donating compound serving as the internal donor.

The amount of the titanium compound used is preferably in a range of from 0.0001 to 1,000, and particularly preferably in a range of from 0.01 to 10, in terms of a molar ratio to the amount of the magnesium compound used (i.e., the molar number of the titanium compound/the molar number of the magnesium compound).

When the halogen compound is used in addition to the magnesium compound and the titanium compound, regardless of whether the magnesium compound and the titanium compound contain a halogen or not, the amount of the halogen compound used is preferably in a range of from 0.01 to 1,000, and particularly preferably in a range of from 0.1 to 100, in terms of a molar ratio to the amount of the magnesium compound used (i.e., the molar number of the halogen compound/the molar number of the magnesium compound).

The amount of the electron donating compound used as the internal donor is preferably in a range of from 0.001 to 10, and particularly preferably in a range of from 0.01 to 5, in terms of a molar ratio to the amount of the magnesium compound used (i.e., the molar number of the electron donating compound/the molar number of the magnesium compound).

After the formation of the solid component (A1), the solid catalyst component may be further brought into contact with the organoaluminum compound (A2), the organosilicon compound (A3) other than a vinylsilane compound, the vinylsilane compound (A4) and so on. For example, after the formation of the solid component (A1), the solid catalyst component can be further brought into contact with the organoaluminum compound (A2) and the organosilicon compound (A3) other than a vinylsilane compound, or after the formation of the solid component (A1), the solid catalyst component can be further brought into contact with the organoaluminum compound (A2), the organosilicon compound (A3) other than a vinylsilane compound, and the vinylsilane compound (A4).

The solid catalyst component (A) containing the (A1), (A2) and (A3) and optionally containing the following (A4) can suppress an excessive decrease in catalytic activity which is caused by the reaction inhibitor.

### (A2: Organoaluminum Compound)

As the organoaluminum compound (A2) used in the solid catalyst component of the present invention, a compound represented by the following general formula (4) is preferably used.

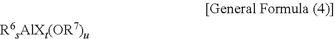

(where R⁶ is a hydrocarbon group; X is a halogen or a hydrogen atom; R⁷ is a hydrocarbon group containing 1 to 20 carbon atoms or a crosslinking group by Al; s, t and u satisfy 1≤s≤3, 0≤t<2 and 0≤u≤2, respectively; and s, t and u satisfy s+t+u=3.)

In the general formula (4), R⁶ is a hydrocarbon group. It is preferably a hydrocarbon group containing 1 to 10 carbon atoms, more preferably a hydrocarbon group containing 1 to 8 carbon atoms, and particularly preferably a hydrocarbon group containing 1 to 6 carbon atoms. As R⁶, examples include, but are not limited to, a methyl group, an ethyl group, a propyl group, a butyl group, an isobutyl group, a hexyl group and an octyl group. Of them, a methyl group, an ethyl group and an isobutyl group are most preferred.

In the general formula (4), X is a halogen or a hydrogen atom. As the halogen that can be used as X, examples include, but are not limited to, fluorine, chlorine, bromine and iodine. Of them, chlorine is particularly preferred.

In the general formula (4), R⁷ is a hydrocarbon group containing 1 to 20 carbon atoms or a crosslinking group by Al. When R⁷ is a hydrocarbon group, R⁷ can be selected from the same group of the hydrocarbon groups exemplified above as R⁶.

Also, an alumoxane compound as typified by methylalumoxane can be used as the organoaluminum compound. In this case, R⁷ represents a crosslinking group by Al.

The crosslinking group by Al denotes an aluminum atom crosslinking two or more residues each having a structure such that R⁷ is removed from the general formula (4), or it denotes an aluminum atom crosslinking a hydrocarbon group and a residue having a structure such that R⁷ is removed from the general formula (4).

As the organoaluminum compound, examples include, but are not limited to, the following: (a) a trialkylaluminum such as trimethylaluminum, triethylaluminum, triisobutylaluminum, tri-n-hexylaluminum, tri-n-octylaluminum and tri-n-decylaluminum, (b) an alkylaluminum halide such as diethylaluminum monochloride, diisobutylaluminum monochloride, ethylaluminum sesquichloride and ethylaluminum dichloride, (c) an alkylaluminum hydride such as diethylaluminum hydride and diisobutylaluminum hydride, and (d) an alkylaluminum alkoxide such as diethylaluminum ethoxide and diethylaluminum phenoxide. Of them, triethylaluminum and triisobutylaluminum are preferred.

As the organoaluminum compound, a single compound can be used, or several compounds can be used in combination.

The amount of the organoaluminum compound used is preferably in a range of from 0.1 to 100, and particularly preferably in a range of from 1 to 50, in terms of the atomic ratio of the aluminum to the titanium (i.e., the molar number of the aluminum atom/the molar number of the titanium atom).

(A3: Organosilicon Compound Other than Vinylsilane Compound)

As the organosilicon compound other than a vinylsilane compound used in the solid catalyst component of the present invention, for example, the compounds disclosed in JP-A No. 2004-124090 can be used, and preferred is an alkoxysilane compound.

As the alkoxysilane compound, a compound represented by the following general formula (5) is preferably used.

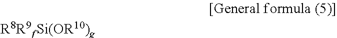

(where R⁸ represents a hydrocarbon group or a heteroatom-containing hydrocarbon group; R⁹ represents a hydrogen atom, a halogen, a hydrocarbon group or a heteroatom-containing hydrocarbon group; R¹⁰ represents a hydrocarbon group; f and g are values satisfying 0≤f≤2 and 1≤g≤3, respectively; and f and g are values satisfying f+g=3.)

In the general formula (5), R⁸ represents a hydrocarbon group or a heteroatom-containing hydrocarbon group. When R⁸ is a hydrocarbon group, it is generally a hydrocarbon group containing 1 to 20 carbon atoms, and preferably a hydrocarbon group containing 3 to 10 carbon atoms. As R⁸, examples include, but are not limited to, a linear aliphatic hydrocarbon group as typified by an n-propyl group, a branched aliphatic hydrocarbon group as typified by an i-propyl group and a t-butyl group, an alicyclic hydrocarbon group as typified by a cyclopentyl group and a cyclohexyl group, and an aromatic hydrocarbon group as typified by a phenyl group. As R⁸, a branched aliphatic hydrocarbon group or an alicyclic hydrocarbon group is more preferably used, and an i-propyl group, an i-butyl group, a t-butyl group, a thexyl group (a 1,1,2-trimethylpropyl group), a cyclopentyl group, a cyclohexyl group or the like is particularly preferably used.

When R⁸ is a heteroatom-containing hydrocarbon group, the heteroatom is preferably selected from nitrogen, oxygen, sulfur, phosphorus and silicon, and the heteroatom is particularly preferably nitrogen or oxygen. As the skeleton structure of the heteroatom-containing hydrocarbon group as R⁸ is preferably selected from those exemplified above for the case where R⁸ is a hydrocarbon group. Of them, an N,N-diethylamino group, a quinolino group, an isoquinolino group or the like is particularly preferred.

When R⁸ is a heteroatom-containing hydrocarbon group, the heteroatom-containing hydrocarbon group may be bound to Si via any one of the carbon atom and heteroatom constituting the heteroatom-containing hydrocarbon group.

In the general formula (5), R⁹ represents a hydrogen atom, a halogen atom, a hydrocarbon group or a heteroatom-containing hydrocarbon group. As the halogen atom that can be used as R⁹, examples include, but are not limited to, fluorine, chlorine, bromine and iodine.

When R⁹ is a hydrocarbon group, it is generally a hydrocarbon group containing 1 to 20 carbon atoms, and preferably a hydrocarbon group containing 1 to 10 carbon atoms. As R⁹, examples include, but are not limited to, a linear aliphatic hydrocarbon group as typified by a methyl group and an ethyl group, a branched aliphatic hydrocarbon group as typified by an i-propyl group and a t-butyl group, an alicyclic hydrocarbon group as typified by a cyclopentyl group and a cyclohexyl group, and an aromatic hydrocarbon group as typified by a phenyl group. Of them, a methyl group, an ethyl group, an n-propyl group, an i-propyl group, an i-butyl group, an s-butyl group, a t-butyl group, a thexyl group, a cyclopentyl group, a cyclohexyl group or the like is preferably used.

When R⁹ is a heteroatom-containing hydrocarbon group, the heteroatom-containing hydrocarbon group is preferably selected from those exemplified above for the case where R⁸ is a heteroatom-containing hydrocarbon group. Of them, an N,N-diethylamino group, a quinolino group, an isoquinolino group or the like is preferred.

When R⁹ is a heteroatom-containing hydrocarbon group, the heteroatom-containing hydrocarbon group may be bound to Si via any one of the carbon atom and heteroatom constituting the heteroatom-containing hydrocarbon group.

When the value of f is 2, two R⁹s may be identical to or different from each other. In spite of the value of f, R⁹ may be identical to or different from R⁸.

In the general formula (5), R¹⁰ represents a hydrocarbon group. R¹⁰ is generally a hydrocarbon group containing 1 to 20 carbon atoms, more preferably a hydrocarbon group containing 1 to 10 carbon atoms, and still more preferably a hydrocarbon group containing 1 to 5 carbon atoms. As R¹⁰, examples include, but are not limited to, a linear aliphatic hydrocarbon group as typified by a methyl group and an ethyl group, and a branched aliphatic hydrocarbon group as typified by an i-propyl group and a t-butyl group. Of them, a methyl group or an ethyl group is preferred. When the value of g is 2 or more, several Rios may be identical to or different from each other.

As the organosilicon compound other than a vinylsilane compound which can be used in the present invention, especially as the alkoxysilane compound, the following are preferred, for example: t-Bu(Me)Si(OMe)², t-Bu(Me)Si(OEt)₂, t-Bu(Et)Si(OMe)₂, t-Bu(n-Pr)Si(OMe)₂, c-Hex(Me)Si(OMe)₂, c-Hex(Et)Si(OMe)₂, c-Pen₂Si(OMe)₂, i-Pr₂Si(OMe)₂, i-Bu₂Si(OMe)₂, i-Pr(i-Bu)Si(OMe)₂, n-Pr(Me)Si(OMe)₂, t-BuSi(OEt)₃, (Et₂N)₂Si(OMe)₂, Et₂N—Si(OEt)₃ and (Et₂N)₂(c-Pen)Si(OMe).

In the above examples, Me represents methyl; Et represents ethyl; t-Bu represents t-butyl; n-Pr represents n-propyl; i-Pr represents isopropyl; c-Hex represents cyclohexyl; and c-Pen represents cyclopentyl.

As the organosilicon compound other than a vinylsilane compound, a single compound can be used, or several compounds can be used in combination.

The amount of the organosilicon compound other than a vinylsilane compound used can be any amount, as long as the effect of the present invention is not impaired. The amount of the organosilicon compound used is preferably in a range of from 0.01 to 1,000, and more preferably in a range of from 0.1 to 100, in terms of the molar ratio of the organosilicon compound to the titanium (the molar number of the organosilicon compound/the molar number of the titanium atom).

The organosilicon compound used in the present invention is considered as follows: the organosilicon compound is coordinated to the vicinity of the titanium atom that can be an active center, such as a Lewis acid site on a magnesium support, and it controls catalytic performance such as catalytic activity and polymer regularity. However, this mechanism of action does not limit the technical scope of the present invention.

### (A4: Vinylsilane Compound)

As the vinylsilane compound used in the solid catalyst component of the present invention, a compound represented by the following general formula (6) is preferably used, which is a compound having a structure such that at least one of the hydrogen atoms of monosilane (SiH₄) is substituted by a vinyl group, and a part or all of the remaining hydrogen atoms are substituted by other free radicals.

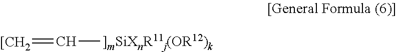

(where X represents a halogen; R¹¹ represents a hydrogen atom or a hydrocarbon group; R¹² represents a hydrogen atom, a hydrocarbon group or an organosilicon group; 1≤m≤4; 0≤n≤3; 0≤j≤3; 0≤k≤2; and m+n+j+k=4.)

In the general formula (6), m denotes the number of the vinyl group and is a value of 1 or more and 4 or less. More preferably, the value of m is desirable to be 1 or 2, and particularly preferably 2.

In the general formula (6), X represents a halogen such as fluorine, chlorine, bromine and iodine. When there are several halogens, they may be identical to or different from each other. Among them, chlorine is particularly preferred. Also, n represents the number of the halogen and is a value of 0 or more and 3 or less. More preferably, the value of n is 0 or more and 2 or less, and particularly preferably 0.

In the general formula (6), R¹¹ represents a hydrogen atom or a hydrocarbon group, and it is preferably a hydrogen atom or a hydrocarbon group containing 1 to 20 carbon atoms, and more preferably a hydrogen atom or a hydrocarbon group containing 1 to 12 carbon atoms. Preferred examples of R¹¹ include, but are not limited to, a hydrogen atom, an alkyl group as typified by a methyl group and a butyl group, a cycloalkyl group as typified by a cyclohexyl group, and an aryl group as typified by a phenyl group. Particularly preferred examples of R¹¹ include, but are not limited to, a hydrogen atom, a methyl group, an ethyl group and a phenyl group. In the general formula (6), j represents the number of R¹¹ and is a value of 0 or more and 3 or less. The value of j is more preferably 1 or more and 3 or less, still more preferably 2 or more and 3 or less, and particularly preferably 2. When j is 2 or more, R¹¹s may be identical to or different from each other.

In the general formula (6), R¹² represents a hydrogen atom, a hydrocarbon group or an organosilicon group. When R¹² is a hydrocarbon group, it can be selected from the same group of the examples provided above as R¹¹. When R¹² is an organosilicon group, it is preferably an organosilicon group containing a hydrocarbon group containing 1 to 20 carbon atoms. As the organosilicon group that can be used as R¹², examples include, but are not limited to, an alkyl group-containing silicon group as typified by a trimethylsilyl group, an aryl group-containing silicon group as typified by a dimethylphenylsilyl group, a vinyl group-containing silicon group as typified by a dimethylvinylsilyl group, and a silicon group formed by combining them, such as a propylphenylvinylsilyl group.

Also, k represents the number of R¹² and is a value of 0 or more and 2 or less. In the case of a compound such that the value of k corresponds to 3 (like vinyltriethoxysilane), the compound does not exert the performance of the vinylsilane compound of the present invention, and it exerts the performance of the alkoxysilane compound of (A3) of the present invention. Accordingly, the compound is not suitable to be used as the vinylsilane compound. The reason for this is considered that the compound functions the same in manner as t-butyltriethoxysilane that is structurally close to the compound (t-butyltriethoxysilane is effective as the organosilicon compound of (A3) of the present invention). The value of k is more preferably 0 or more and 1 or less, and particularly preferably 0. When the value of k is 2, two R¹²s may be identical to or different from each other. Regardless of the value of k, R¹¹ and R¹² may be identical to or different from each other.

These vinylsilane compounds can be used alone or in combination.

Preferred examples of the vinylsilane compound include, but are not limited to, CH₂═CH—SiMe₃, [CH₂═CH—]₂SiMe₂, CH₂═CH—Si(Cl)Me₂, CH═CH—Si(Cl)₂Me, CH═CH—SiCl₃, [CH₂═CH—]₂Si(Cl)Me, [CH₂═CH—]₂SiCl₂, CH₂═CH—Si(Ph)Me₂, CH₂═CH—Si(Ph)₂Me, CH₂═CH—SiPh₃, [CH₂═CH—]₂Si(Ph)Me, [CH₂═CH—]₂SiPh₂, CH₂═CH—Si(H)Me₂, CH₂═CH—Si(H)₂Me, CH₂═CH—SiH₃, [CH₂═CH—]₂Si(H)Me, [CH₂═CH—]₂SiH₂, CH₂═CH—SiEt₃, CH₂═CH—SiBu₃, CH₂═CH—Si(Ph)(H)Me, CH₂═CH—Si(Cl)(H)Me, CH₂═CH—Si(Me)₂(OMe), CH₂═CH—Si(Me)₂(OSiMe₃) and CH₂CH—Si(Me)₂-OSi(Me)₂-CH═CH₂. Of them, preferred is a divinylsilane compound such that m=2, and particularly preferred is divinyldimethylsilane ([CH₂═CH—]₂SiMe₂).

In the above examples, Ph represents a phenyl group, and other symbols such as Me, Et and Bu are as described above.

The amount of the vinylsilane compound used can be any amount, as long as the effect of the present invention is not impaired. The amount of the vinylsilane compound used is, for example, in a range of from 0 to 1000, preferably in a range of from 0.001 to 1000, and particularly preferably in a range of from 0.01 to 100, in terms of the molar ratio of the vinylsilane compound to the titanium (the molar number of the vinylsilane compound/the molar number of the titanium atom).

The vinylsilane compound used in the present invention is considered as follows: the charge density of the carbon-carbon double bond moiety is very high, and coordination to the titanium atom, which is an active center, is very fast; therefore, the vinylsilane compound is effective in preventing the overreduction of the titanium atom by the organoaluminum compound and preventing the deactivation of the active site by impurities or the like. However, this mechanism of action does not limit the technical scope of the present invention.

### (Solid Catalyst Component Preparing Method)

The solid catalyst component used in the present invention is obtained by bringing the solid catalyst component into contact with the above-described components and forming the solid component.

A condition for bringing the solid catalyst component into contact with the above-described components, needs to be the absence of oxygen. However, any condition can be employed as long as the effect of the present invention is not impaired. In general, the following conditions are preferred.

The contact temperature from about −50° C. to about 200° C., and it is preferably from 0° C. to 150° C.

As the contact method, examples include, but are not limited to, a mechanical method using a tumbling ball mill or a vibrating mill, and a contact method in the presence of an inert diluent by stirring.

In the preparation of the solid catalyst component, washing with an inert solvent may be carried out in the middle of and/or at the end of the preparation.

The inert solvent is, for example, preferably an aliphatic hydrocarbon compound such as heptane, an aromatic hydrocarbon compound such as toluene and xylene, or a halogen-containing hydrocarbon compound such as 1,2-dichloroethylene and chlorobenzene.

As the solid catalyst component preparing method, any method can be used. As the method, examples include, but are not limited to, the methods described below as (i) to (viii).

### (i) Co-Milling Method

The co-milling method is a method for supporting a titanium compound on a magnesium compound by co-milling a halogen-containing magnesium compound (as typified by magnesium chloride) and a titanium compound. In this method, an electron donating compound may be co-milled at the same time or in a different step.

Also, a dry milling method without a solvent, a wet milling method in which co-milling is carried out in the coexistence of an inert solvent, or the like can be used. For the milling, any mill such as a tumbling ball mill and a vibrating mill can be used.

### (ii) Heat Treatment Method

The heat treatment method is a method for supporting a titanium compound on a halogen-containing magnesium compound (as typified by magnesium chloride) by bringing them in contact with each other by stirring and heating them in an inert solvent. In this method, an electron donating compound may be brought into contact with them at the same time or in a different step.

When a liquid compound such as titanium tetrachloride is used as the titanium compound, they can be brought into contact with each other without an inert solvent.

As needed, an optional component such as a halogenated silicon compound may be brought into contact with them at the same time or in a different step.

The contact temperature is not particularly limited. In many cases, they are preferably brought into contact with each other at a relatively high temperature of from about 90° C. to about 130° C.

(iii) Dissolution-Precipitation Method

The dissolution-precipitation method is a method for forming particles by dissolving a halogen-containing magnesium compound (as typified by magnesium chloride) by bringing it into contact with an electron donating compound, and then causing a precipitation reaction by bringing the thus-produced solution into contact with a precipitant.

Among the electron donating compounds described above, those that can be used for the dissolution are, for example, alcohols and ethers.

As the precipitant, examples include, but are not limited to, a halogenated titanium compound, a halogenated silicon compound, hydrogen chloride, a halogen-containing hydrocarbon compound, a siloxane compound containing a Si—H bond (including a polysiloxane compound) and an aluminum compound.

To bring the thus-produced solution and the precipitant into contact with each other, the precipitant may be added to the thus-produced solution, or the thus-produced solution may be added to the precipitant.

When a titanium compound is not used in both the dissolution step and the precipitation step, the particles formed by the precipitation reaction are further brought into contact with a titanium compound, thereby supporting the titanium compound on the magnesium compound.

In addition, as needed, the particles formed by the above method may be brought into contact with an optional component such as a halogenated titanium compound and a halogenated silicon compound, or they may be brought into contact with an electron donating compound. In this case, the electron donating compound may be different from or the same as the electron donating compound used for the dissolution.

These optional components may be brought into contact in any order, without particular limitation. They may be brought into contact with each other in an independent step, or they can be brought into contact with each other during the dissolution, during the precipitation, or during the contact with the titanium compound and so on.

Also, an inert solvent may be present in all of the dissolution step, the precipitation step, and the step of being brought into contact with the optional components.

### (iv) Granulation Method

The granulation method is a method of granulation by, as with the dissolution-precipitation method, dissolving a halogen-magnesium compound (as typified by magnesium containing chloride) by bringing it into contact with an electron donating compound, and then granulating the thus-produced solution by mainly a physical method. The examples of the electron donating compound used for the dissolution are the same as the examples provided in the dissolution-precipitation method.

As the granulation method, examples include, but are not limited to, a method of adding the solution, which is at high temperature, to the inert solvent, which is at low temperature, in a dropwise manner, a method of ejecting the solution from a nozzle to a high-temperature vapor phase and drying the ejected solution, and a method of ejecting the solution from a nozzle to a low-temperature vapor phase and cooling the ejected solution.

The particles formed by the granulation are brought into contact with a titanium compound, thereby supporting the titanium compound on the magnesium compound.

In addition, as needed, the particles may be brought into contact with an optional component such as a halogenated silicon compound and an electron donating compound. In this case, the electron donating compound may be different from or the same as the electron donating compound used for the dissolution.

These optional components may be brought into contact in any order, without particular limitation. They may be brought into contact with each other in an independent step, or they can be brought into contact with each other during the dissolution or during the contact with the titanium compound.

Also, an inert solvent may be present in all of the dissolution step, the step of being brought into contact with the titanium compound, and the step of being brought into contact with the optional components.

### (v) Magnesium (Mg) Compound Halogenation Method

The magnesium (Mg) compound halogenation method is a method for halogenating a halogen-free magnesium compound by bringing the compound into contact with a halogenating agent. In this method, an electron donating compound may be brought into contact with them at the same time or in a different step.

As the halogen-free magnesium compound, examples include, but are not limited to, a dialkoxymagnesium compound, magnesium oxide, magnesium carbonate, and magnesium salt of fatty acid.

When a dialkoxymagnesium compound is used, a dialkoxymagnesium compound prepared by reacting magnesium metal with an alcohol in situ, can be used. When this preparation method is used, generally, particle formation is carried out by granulation or the like at the stage of a halogen-free magnesium compound (a starting material).

As the halogenating agent, examples include, but are not limited to, a halogenated titanium compound, a halogenated silicon compound and a halogenated phosphorus compound.

When a halogenated titanium compound is not used as the halogenating agent, the halogen-containing magnesium compound formed by halogenation is further brought into contact with a titanium compound, thereby supporting the titanium compound on the magnesium compound.

In addition, as needed, the particles formed by the above method may be brought into contact with an optional component such as a halogenated titanium compound and a halogenated silicon compound, or they may be brought into contact with an electron donating compound.

These optional components may be brought into contact in any order, without particular limitation. They may be brought into contact with each other in an independent step, or they can be brought into contact with each other during the halogenation of the halogen-free magnesium compound, or during the contact with the titanium compound.

Also, an inert solvent may be present in all of the step of being brought into contact with the halogenated titanium compound and the step of being brought into contact with the optional components.

(vi) Precipitation Method from Organomagnesium Compound

The precipitation method from an organomagnesium compound is a method of bringing a precipitant into contact with a solution of an organomagnesium compound such as a Grignard reagent (as typified by butylmagnesium chloride) and a dialkylmagnesium compound. In this method, an electron donating compound may be brought into contact with them at the same time or in a different step.

As the precipitant, examples include, but are not limited to, a titanium compound, a silicon compound and hydrogen chloride.

When a titanium compound is not used as the precipitant, the particles formed by the precipitation reaction are further brought into contact with a titanium compound, thereby supporting the titanium compound on the magnesium compound.

In addition, as needed, the particles formed by the above method may be brought into contact with an optional component such as a halogenated titanium compound and a halogenated silicon compound, or they may be brought into contact with an electron donating compound.

These optional components may be brought into contact in any order, without particular limitation. They may be brought into contact with each other in an independent step, or they can be brought into contact with each other during the precipitation or during the contact with the titanium compound.

Also, an inert solvent may be present in all of the precipitation step, the step of being brought into contact with the titanium compound, and the step of being brought into contact with the optional components.

(vii) Impregnation Method

The impregnation method is a method for impregnating a support composed of an inorganic or organic compound with a solution of an organomagnesium compound or a solution obtained by dissolving a magnesium compound by an electron donating compound.

Examples of the organomagnesium compound are the same as those of the above-described precipitation method from the organomagnesium compound. The magnesium compound used to dissolve the magnesium compound may contain or may be halogen-free. Examples of the electron donating compound are the same as those of the above-described dissolution-precipitation method.

As the support composed of the inorganic compound, examples include, but are not limited to, silica, alumina and magnesia.

As the support composed of the organic compound, examples include, but are not limited to, polyethylene, polypropylene and polystyrene.

On the support particles after the impregnation treatment, the magnesium compound is precipitated and fixed by a chemical reaction with a precipitant or by a physical treatment such as drying.

Examples of the precipitant are the same as those of the above-described dissolution-precipitation method.

When a titanium compound is not used as the precipitant, the particles formed in this manner are further brought into contact with a titanium compound, thereby supporting the titanium compound on the magnesium compound. In addition, as needed, the particles formed in this manner may be brought into contact with an optional component such as a halogenated titanium compound and a halogenated silicon compound, or they may be brought into contact with an electron donating compound.

These optional components may be brought into contact in any order, without particular limitation. They may be brought into contact with each other in an independent step, or they can be brought into contact with each other during the impregnation, during the precipitation, during the drying, or during the contact with the titanium compound. Also, an inert solvent may be present in all of the impregnation step, the precipitation step, the step of being brought into contact with the titanium compound, and the step of being brought into contact with the optional components.

(viii) Combined Method

The above-described methods (i) to (vii) can be combined and used. As the combination of the methods, examples include, but are not limited to, the following: “a method of co-milling magnesium chloride and the electron-donating compound, followed by heat treatment with the halogenated titanium compound”, “a method of co-milling magnesium chloride and the electron-donating compound, followed by dissolution with another electron-donating compound and then precipitation with the precipitant”, “a method of dissolving the dialkoxymagnesium compound by the electron-donating compound, precipitating them by contact with the halogenated titanium compound, and simultaneously halogenating the magnesium compound” and “a method in which, by bringing the dialkoxymagnesium compound into contact carbon dioxide, a carbonate ester magnesium compound is formed and dissolved at the same time; silica is impregnated with the thus-produced solution; and the impregnated silica is brought into contact with hydrogen chloride, thereby halogenating the magnesium compound and, at the same time, precipitating and fixing the halogenated magnesium compound; and then the precipitated and fixed halogenated magnesium compound is brought into contact with the halogenated titanium compound, thereby supporting the titanium compound.”

When the solid catalyst component is a solid catalyst component obtained by, after the formation of the solid component (A1), further bringing the solid component (A1) into contact with the organoaluminum compound (A2), the organosilicon compound (A3) other than a vinylsilane compound, the vinylsilane compound (A4) and so on, the method for bringing the components into contact with each other is not particularly limited. In general, each of the above components can be brought into contact with one another while stirring the components in the presence of an inert solvent.

As the inert solvent, examples include, but are not limited to, liquid saturated hydrocarbon such as hexane, heptane, octane, decane, dodecane and liquid paraffin, and silicon oil having the structure of dimethylpolysiloxane. These inert solvents may be used alone, or they may be used in combination of two or more kinds as a mixed solvent. The inert solvent is preferably used after impurities such as oxygen, water and a sulfur compound, which have a negative influence on polymerization, are removed therefrom.

The contact condition may be any condition, as long as the effect of the present invention is not impaired. The contact temperature is generally from about −50° C. to about 200° C., preferably from −10° C. to 100° C., more preferably from 0° C. to 70° C., and still more preferably from 10° C. to 60° C.

When the solid catalyst component is a solid catalyst component obtained by, after the formation of the solid component (A1), further bringing the solid component (A1) into contact with the organoaluminum compound (A2) and the organosilicon compound (A3) other than a vinylsilane compound, any procedure can be used as the contact procedure of the solid component (A1) with the organoaluminum compound (A2) and the organosilicon compound (A3) other than a vinylsilane compound. As the procedure, examples include, but are not limited to, the following procedures (i) to (iv). Of them, the procedures (i) and (ii) are preferred.

Procedure (i): A method of bringing the solid component (A1) into contact with the organosilicon compound (A3) other than a vinylsilane compound and then with the organoaluminum compound (A2).

Procedure (ii): A method of bringing the solid component (A1) into contact with the organoaluminum compound (A2) and then with the organosilicon compound (A3) other than a vinylsilane compound.

Procedure (iii): A method of bringing the organosilicon compound (A3) other than a vinylsilane compound into contact with the organoaluminum compound (A2) and then with the solid component (A1).

Procedure (iv): A method in which all the components are brought into contact with each other at the same time.

The number of times of bringing the solid component (A1), the organoaluminum compound (A2) and the organosilicon compound (A3) other than a vinylsilane compound into contact with each other, may be any number. In this case, the components that are used several times may be the same as or different from each other.

The preferred range of the amount of each of the components used, is as described above. This is the amount of each component used per contact. If the component is brought into contact several times, it may be brought into contact any number of times, on the basis that the amount of the component used per contact falls into the above-described amount range.

When the solid catalyst component is brought into contact with other components, any contact method, any contact condition and any contact procedure can be employed.

When the solid catalyst component is a solid catalyst component obtained by, after the formation of the solid component (A1), further bringing the solid component (A1) into contact with the organoaluminum compound (A2), the organosilicon compound (A3) other than a vinylsilane compound and the vinylsilane compound (A4), any procedure can be used as the contact procedure of the solid component (A1) with the organoaluminum compound (A2), the organosilicon compound (A3) other than a vinylsilane compound and the vinylsilane compound (A4). As the procedures, examples include, but are not limited to, the following procedures (iv) to (vii). Of them, the procedures (iv) and (v) are preferred.

Procedure (iv): A method of bringing the solid component (A1) into contact with the vinylsilane compound (A4), with the organosilicon compound (A3) other than a vinylsilane compound, and then with the organoaluminum compound (A2).

Procedure (v): A method of bringing the organosilicon compound (A3) other than a vinylsilane compound into contact with the vinylsilane compound (A4), with the solid component (A1), and then with the organoaluminum compound (A2).

Procedure (vi): A method of bringing the solid component (A1) into contact with the vinylsilane compound (A4) and then with the organosilicon compound (A3) other than a vinylsilane compound and the organoaluminum compound (A2).

Procedure (vii): A method in which all the components are brought into contact with each other at the same time.

The number of times of bringing the solid component (A1), the organoaluminum compound (A2), the organosilicon compound (A3) other than a vinylsilane compound and the vinylsilane compound (A4) into contact with each other, may be any number. In this case, the components that are used several times may be the same as or different from each other.

The preferred range of the amount of each of the components used, is as described above. This is the amount of each component used per contact. If the component is brought into contact several times, it may be brought into contact any number of times, on the basis that the amount of the component used per contact falls into the above-described amount range.

When the solid catalyst component is brought into contact with other components, any contact method, any contact condition and any contact procedure can be employed.

### (Preliminary Polymerization of the Solid Catalyst Component)

The solid catalyst component may be preliminarily polymerized. In the presence of the solid catalyst component, a small amount of a compound containing an ethylenic double bond is polymerized in a mild condition as a monomer (a monomer for preliminary polymerization). Accordingly, a part or the whole of the monomer for preliminary polymerization is polymerized into a polymer (preliminarily polymerized polymer) of the compound containing the ethylenic double bond, which can be a solid catalyst component suitable for the polymerization of propylene-based block copolymers.

As the monomer for preliminary polymerization, examples include, but are not limited to, an olefin as typified by ethylene, propylene, 1-butene, 3-methylbutene-1, 4-methylpentene-1, 1-pentene, 1-hexene, 1-octene, 1-decene, 1-dodecene, 1-tetradecene, 1-hexadecene, 1-octadecene, 1-eicosene, 4-methyl-1-pentene and 3-methyl-1-pentene; a styrene analog as typified by styrene, α-methylstyrene, allylbenzene and chlorostyrene; and a diene compound as typified by 1,3-butadiene, isoprene, 1,3-pentadiene, 1,5-hexadiene, 2,6-octadiene, dicyclopentadiene, 1,3-cyclohexadiene, 1,9-decadiene and a divinylbenzene. Of them, preferred are ethylene, propylene, 3-methylbutene-1, 4-methylpentene-1, styrene and a divinylbenzene. They may be used alone or in combination of two or more kinds.

To control the molecular weight of the polymer produced by the preliminary polymerization, a molecular weight regulator such as hydrogen may be used in combination.

The solid catalyst component obtained by the preliminary polymerization contains the polymer (preliminarily polymerized polymer) of the compound containing the ethylenic double bond. When propylene is homopolymerized or copolymerized by use of the solid catalyst component, the preliminary polymerized polymer functions as a shell. Accordingly, the effect of suppressing fine powder production, which is due to broken catalyst particles, in the main polymerization, is obtained.

From the viewpoint of producing a sufficient amount of the preliminarily polymerized polymer in the preliminary polymerization process, the amount of the monomer for preliminary polymerization used is preferably 0.1 parts by mass or more, more preferably 0.2 parts by mass or more, still more preferably 0.4 parts by mass or more, and even more preferably 0.5 parts by mass or more, per part by mass of the solid catalyst component before being subjected to the preliminary polymerization.

The upper limit of the amount of the monomer for preliminary polymerization used is not particularly limited. From the viewpoint of preventing an excessive increase in the amount of the preliminarily polymerized polymer thus produced, the amount of the monomer for preliminary polymerization is preferably 20 parts by mass or less, more preferably 15 parts by mass or less, and still more preferably 10 parts by mass or less, per part by mass of the solid catalyst component before being subjected to the preliminary polymerization.

The amount of the preliminarily polymerized polymer contained in the preliminarily polymerized solid catalyst component, that is, the preliminarily polymerized amount, is preferably 0.1 parts by mass or more, more preferably 0.2 parts by mass or more, still more preferably 0.4 parts by mass or more, and even more preferably 0.5 parts by mass or more, per part by mass of the solid catalyst component before being subjected to the preliminary polymerization. When the preliminarily polymerized amount is in the above-described range, the effect of suppressing fine powder production, which is due to broken catalyst particles, is obtained.

The upper limit of the preliminarily polymerized amount is not particularly limited. From the viewpoint of productivity and economic efficiency, the upper limit of the preliminarily polymerized amount is preferably 20 parts by mass or less, more preferably 15 parts by mass or less, and still more preferably 10 parts by mass or less, per part by mass of the solid catalyst component before being subjected to the preliminary polymerization. This is because catalyst performance peaks out even if the preliminarily polymerized amount is increased above the range.

The preliminary polymerization method is not particularly limited. In general, preliminary polymerization is carried out while stirring the components in the presence of an inert solvent. As the inert solvent, examples include, but are not limited to, liquid saturated hydrocarbon such as hexane, heptane, octane, decane, dodecane and liquid paraffin, and silicon oil having the structure of dimethylpolysiloxane. These inert solvents may be used alone, or they may be used in combination of two or more kinds as a mixed solvent. The inert solvent is preferably used after impurities such as oxygen, water and a sulfur compound, which have a negative influence on polymerization, are removed therefrom.

The preliminary polymerization condition may be any condition, as long as the effect of the present invention is not impaired.

The preliminary polymerization reaction temperature is generally from about −50° C. to about 200° C., preferably from −10° C. to 100° C., and more preferably from 0° C. to 70° C.

The preliminary polymerization may be carried out in the presence of an organoaluminum compound. As the organoaluminum compound, examples include, but are not limited to, those exemplified above as the organoaluminum compound (A2).

The amount of the organoaluminum compound used in the preliminary polymerization step is in a range of from 0.1 moles to 40 moles, and preferably in a range of from 0.3 moles to 20 moles, per mole of the titanium atom of the solid catalyst component.

Also, the preliminary polymerization may be carried out in the presence of an alkoxysilane compound. As the alkoxysilane compound, examples include, but are not limited to, those exemplified above as the organosilicon compound (A3) other than a vinylsilane compound. The amount of the alkoxysilane compound used in the preliminary polymerization step is preferably in a range of from 0.01 moles to 10 moles per mole of the titanium contained in the solid catalyst component.

The preliminary polymerization may be carried out in several batches. The monomers for preliminarily polymerization used in this case may be identical to or different from each other. Also, washing with an inert solvent such as hexane and heptane can be carried out after the preliminary polymerization. After the preliminary polymerization is completed, depending on the form of usage of the catalyst, the catalyst can be continuously used as it is, or it may be dried.

Also, as long as the effect of the present invention is not impaired, an optional component may be added in the middle of the washing or drying after the preliminary polymerization, or it may be added after the washing or drying. As the optional component, examples include, but are not limited to, a polymer such as polyethylene, polypropylene and polystyrene, and a solid inorganic oxide such as silica and titania.

### 2. Component (B): Organoaluminum Compound

In the present invention, as the organoaluminum compound (B) that can be used in the olefin polymerization catalyst during the main polymerization, examples include, but are not limited to, the compounds disclosed in JP-A No. 2004-124090. The organoaluminum compound (B) can be preferably selected from the same group of the examples of the organoaluminum compound (A2) which is provided above as a component used in the preparation of the solid catalyst component (A).

The organoaluminum compound (B) may be identical to or different from the organoaluminum compound (A2) used in the preparation of the solid catalyst component (A).

As the organoaluminum compound (B), one kind of compound or a combination of two or more kinds of compounds can be used.

The amount of the organoaluminum compound (B) used is preferably in a range of from 1 to 5,000, and particularly preferably in a range of from 10 to 500, in terms of a molar ratio to the titanium component constituting the solid catalyst component (A) (i.e., the molar number of the organoaluminum compound/the molar number of the titanium atom in the solid catalyst component).

### 3. Electron Donating Compound Serving as External Donor

In the present the olefin polymerization catalyst may include, in its constitutional components, an electron donating compound serving as an external donor.

In the polymerization technique using a Ziegler catalyst, as described above, the external donor changes the nature of an already-formed active site. For example, by further using the external donor for the prepared solid catalyst component, the already-formed active site can be changed into a highly stereospecific active site, and an active site that can produce an amorphous component can be poisoned. Accordingly, a propylene-based polymer which has higher stereoirregularity and in which the content of the amorphous component is less, can be produced.

As the electron donating compound (external donor), examples include, but are not limited to, an organosilicon compound (C), a compound (D) containing at least two ether bonds, a compound (E) containing a C(═O)N bond in the molecule, and a sulfite ester compound (F). As the electron donating compound, one kind of compound or a combination of two or more kinds of compounds can be used.

### (Organosilicon Compound (C))

As the organosilicon compound (C), the compounds disclosed in JP-A No. 2004-124090 can be used. The organosilicon compound (C) can be preferably selected from the same group of the examples of the organosilicon compound (A3) used in the preparation of the solid catalyst component (A).

The organosilicon compound (C) may be identical to or different from the organosilicon compound (A3) other than a vinylsilane compound, which is used in the preparation of the solid catalyst component (A).

As the organosilicon compound (C), one kind of compound or a combination of two or more kinds of compounds can be used.

### (Compound (D) Containing at Least Two Ether Bonds)

As the compound (D) containing at least two ether bonds, for example, the compounds disclosed in JP-A No. H03-294302 and H08-333413 can be used. In general, it is desirable to use the compound represented by the following formula.

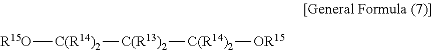

(where R¹³ and R¹⁴ each represent a hydrogen atom, a hydrocarbon group or an optional free radical selected from heteroatom-containing hydrocarbon groups, and R¹⁵ represents a hydrocarbon group or a heteroatom-containing hydrocarbon group.)

As the compound containing at least two ether bonds, examples include, but are not limited to, 2,2-diisopropyl-1,3-dimethoxypropane, 2,2-diisobutyl-1,3-dimethoxypropane, 2-isobutyl-2-isopropyl-1,3-dimethoxypropane, 2-isopropyl-2-isopentyl-1,3-dimethoxypropane, 2,2-dicyclopentyl-1,3-dimethoxypropane, and 9,9-bis(methoxymethyl)fluorene. As the compound containing at least two ether bonds, a single compound can be used, or several compounds can be used in combination.

### (Compound (E) Containing a C(═O)N Bond in the Molecule)

As the compound (E) containing a C(═O)N bond in the molecule, for example, the compounds disclosed in JP-A No. 2004-124090 can be used. Preferred are tetramethylurea, 1,3-dimethyl-2-imidazolidinone and 1-ethyl-2-pyrrolidinone, for example.

### (Sulfite Ester Compound (F))

As the sulfite ester compound, for example, the compounds disclosed in JP-A No. 2006-225449 can be used. Preferred are dimethyl sulfite and diethyl sulfite, for example.

The amount of the electron donating compound (external donor) used is preferably in a range of from 0.01 to 10,000, and particularly preferably in a range of from 0.5 to 500, in terms of a molar ratio to the titanium constituting the solid catalyst component (i.e., the molar number of the electron donating compound/the molar number of the titanium atom in the solid catalyst component).

### III. Produced Propylene-Based Polymer

In the propylene-based polymer produced by the present invention, the melt flow rate (MFR) of the first propylene-based polymer can be controlled by using, during the homopolymerization of the propylene produced by the first step or during the copolymerization of the propylene and another α-olefin, a molecular weight modifier such as hydrogen in the polymerization step. The MFR of the propylene-based polymer is determined by the molding method or by the intended application. The MFR value (unit: g/10 min) measured in the measurement conditions of 230° C. and a load of 2.16 kg is generally 0.1 or more, preferably 0.5 or more, and more preferably 1 or more. On the other hand, it is 500 or less, preferably 400 or less, and more preferably 300 or less. When the MFR is too small, polymer flowability remarkably deteriorates and makes molding difficult. When the MER is too large, a reduction in tensile properties occurs.

In the present invention, the first propylene-based polymer means a propylene homopolymer or a copolymer of propylene and a comonomer. As the comonomer, at least one kind selected from the group consisting of linear and branched α-olefins each containing 2 to 10 carbon atoms other than propylene, can be used. In general, ethylene or 1-butene is preferred. The content of the comonomer is preferably in a range of from 0% by mass to 10% by mass, more preferably in a range of from 0% by mass to 6% by mass, and still more preferably in a range of from 0% by mass to 4% by mass. When the comonomer content is outside the range, the generation of a component too low in crystallinity increases, which may lead to easy formation of lump polymers due to melting or fusion during the polymerization reaction.

In the second step, the intrinsic viscosity [η] of the propylene-based polymer can be controlled by using a molecular weight modifier such as hydrogen in the polymerization step. From the viewpoint of properties such as an increase in melt tension and an improvement in flow mark, and from the viewpoint of improving product appearance by suppressing the number of gels, the intrinsic viscosity [η] of the propylene-ethylene copolymer is preferably in a range of from 2 dL/g to 12 dL/g, and more preferably in a range of from 2.5 dL/g to 10 dL/g.

In the present invention, the second propylene-based polymer may be a propylene homopolymer or a copolymer with a comonomer. As the comonomer, at least one kind selected from the group consisting of linear and branched α-olefins each containing 2 to 10 carbon atoms other than propylene, can be used. In general, ethylene or 1-butene is preferred. The content of the comonomer is preferably in a range of from 0% by mass to 90% by mass, more preferably in a range of from 0% by mass to 70% by mass, and still more preferably in a range of from 0% by mass to 50% by mass. When the comonomer content is outside the range, the compatibility of the first propylene-based polymer and the second propylene-based polymer with each other may decrease, which may lead to a decrease in the quality of the final product, such as impact resistance. Also, the comonomer content of the first propylene-based polymer and that of the second propylene-based polymer may be equal to or different from each other.

From the viewpoint of finely dispersing the components produced in the polymerization steps and keeping the quality of the final product (such as appearance and impact resistance) high, the second propylene-based polymer produced in the second step is preferably a copolymer of propylene and at least one kind of monomer selected from the group consisting of α-olefins containing 2 to 10 carbon atoms other than propylene, and the content of the at least one kind of monomer selected from the group consisting of α-olefins containing 2 to 10 carbon atoms other than propylene, is preferably in a range of from 20% by mass to 80% by mass, and more preferably in a range of from 30% by mass to 70% by mass.

When the whole propylene-based polymer is 100% by mass, it is preferable that the first propylene-based polymer is from 98% by mass to 40% by mass, and the second propylene-based polymer is from 2% by mass to 60% by mass; it is more preferable that the second propylene-based polymer is from 3% by mass to 55% by mass, and the first propylene-based polymer is from 97% by mass to 45% by mass; and it is still more preferable that the second propylene-based polymer is from 3% by mass to 45% by mass, and the first propylene-based polymer is from 97% by mass to 55% by mass.

### EXAMPLES

Hereinafter, the present invention will be described in more detail, with reference to examples and comparative examples. However, the present invention is not limited to these examples. The method for measuring physical values in the present invention is shown below.

### [Measurement of Various Kinds of Physical Properties]

### (1) MFR

The MFR of the propylene-based polymer obtained in each of Examples was evaluated in the conditions according to JIS K7210 (at 230° C. under a load of 2.16 kg).

### (2) Analysis Methods of Propylene-Based Block Copolymer

In the case of producing a so-called propylene-based block copolymer by polymerizing, in at least one of the first and second steps, a propylene-ethylene random copolymer having a large comonomer content and not showing an obvious melting point, the ratio (Wc) and ethylene content (Gv) of the propylene-based copolymer portion and intrinsic viscosity [η] were measured using the below-described cross fractionation device and conditions.

When the comonomer content of the first step and that of the second step were both small and the polymers or the comonomers had an obvious melting point, the production amount in each polymerization step was calculated from the flow rate of a refrigerant supplied for removal of polymerization reaction heat in each polymerization step and a difference in temperature between the inlet and the outlet. Then, the production rate in the second step was obtained by the following calculation formula: [the production amount in the second step]/([the production amount in the first step]+[the production amount in the second step])×100.

### (2-1) Analyzers Employed

### (i) Apparatus for Cross Fractionation

“CFC T-100” manufactured by DIA Instruments Co., Ltd. (hereinafter abbreviated as “CFC”)

### (ii) Fourier Transform Infrared Absorption Spectroscopy

FT-IR “1760X” manufactured by Perkin Elmer, Inc.

Instead of a fixed wavelength infrared spectrophotometer installed as a detector of the CFC, the FT-IR is connected. This FT-IR is used as a detector. A transfer line from an outlet of the solution eluted from the CFC to the FT-IR is set to 1 m and kept at 140° C. throughout the measurement. A flow cell installed in the FT-IR has an optical path length of 1 mm and an optical path width of 5 mm q, and it is kept at 140° C. throughout the measurement.

(iii) Gel Permeation Chromatography (GPC):

As a GPC column in the latter stage part of the CFC, three “AD806 MS” columns manufactured by Showa Denko K.K. are connected in series are used.

### (2-2) Measurement Conditions of CFC

(i) Solvent: Orthodichlorobenzene (ODCB)
(ii) Sample concentration: 4 mg/mL
(iii) Injection amount: 0.4 mL
(iv) Crystallization: Temperature is lowered from 140° C. to 40° C. in about 40 minutes.
(v) Fractionation method:

The fractionation temperature at the time of temperature-rising elution fractionation is set at 40, 100 and 140° C. and fractionated into three fractions in total. The elution ratio (unit: % by mass) of a component eluted at 40° C. or less (fraction 1), that of a component eluted at a temperature of from 40° C. to 100° C. (fraction 2), and that of a component eluted at a temperature of from 100° C. to 140° C. (fraction 3) are defined as W40, W100 and W140, respectively. W40+W100+W140=100. The fractionated fractions are each automatically transported to the FT-IR analyzer as they are.
(vi) Solvent flow rate at the time of elution: 1 mL/min

### (2-3) Measurement Condition of FT-IR

After the elution of the sample solution is started from the GPC of the latter stage of the CFC, the FT-IR measurement is carried out under the following conditions, and GPC-IR data on the above-described fractions 1 to 3 are collected.
(i) Detector: MCT
(ii) Resolution: 8 cm⁻¹
(iii) Measurement interval: 0.2 minute (12 seconds)
(vi) Accumulation times per measurement: 15 times

### (2-4) Post Treatment and Analysis of Measurement Results

The elution amount and molecular weight distribution of the component eluted at each temperature are determined using an absorbance at 2945 cm⁻¹ obtained by FT-IR as a chromatogram. The elution amount is normalized such that the sum of the elution amounts of the individual eluted components is 100%. Conversion from the retention volume to the molecular weight is carried out using a calibration curve prepared in advance with standard polystyrenes.

The standard polystyrenes used are products with following trade names manufactured by Tosoh Corporation: F380, F288, F128, F80, F40, F20, F10, F4, F1, A5000, A2500 and A1000.

A calibration curve is prepared by injecting 0.4 mL of a solution prepared by dissolving each standard polystyrene in ODCB to give a concentration of 0.5 mg/mL (containing 0.5 mg/mL of BHT). As the calibration curve, a cubic expression obtained by approximation by the least squares method is employed. For the conversion to the molecular weight, a universal calibration curve is employed with reference to Sadao Mori, “Size Exclusion Chromatography”, (Kyoritsu Shuppan Co., Ltd.) For the viscosity expression used herein ([η]=K×Mα), the following numerical values are used.
(i) At the time of preparation of the calibration curve using the standard polystyrene: K=0.000138, α=0.70
(ii) At the time of measurement of a sample of the propylene-based block copolymer sample: K=0.000103, α=0.78

The ethylene content distribution of each of the eluted components (the distribution of the ethylene content along the molecular weight axis) is determined by converting the ratio of the absorbance at 2956 cm⁻¹ to the absorbance at 2927 cm⁻¹, both of which are obtained by FT-IR, into an ethylene content (% by mass) by use of a calibration curve prepared in advance using polyethylene, polypropylene, ethylene-propylene rubber (EPR) whose ethylene content is known by ¹³C-NMR measurement, or a mixture thereof.

### (2-5) Ratio (Wc) of Propylene-Ethylene Random Copolymer Portion

The ratio (Wc) of the propylene-ethylene random copolymer portion in the propylene-based block copolymer of the present invention is defined, in theory, by the below-described formula

(I) and determined by the below-described procedures.

$$Wc ( \% by mass ) = {W}_{40} \times {A}_{40} / {B}_{40} + {W}_{100} \times {A}_{100} / {B}_{100} \qquad ( I )$$

In the formula (I), W₄₀ and W₁₀₀ each represent an eluted proportion (unit: % by mass) at each of the above-described fractions; A₄₀ and A₁₀₀ each represent an average ethylene content (unit: % by mass) measured at each of the fractions corresponding to W₄₀ and W₁₀₀, respectively; and B₄₀ and B₁₀₀ each represent an ethylene content (unit: % by mass) of the propylene-ethylene random copolymer portion contained in each of the fractions. These A₄₀, A₁₀₀, B₄₀ and B₁₀₀ will be determined in the manner described later.

The meaning of the formula (I) is as follows. Described specifically, the first term on the right side of the formula (I) is a term for calculating the amount of propylene-ethylene random copolymer portion contained in the fraction 1 (a portion soluble at 40° C.). When the fraction 1 does not contain a crystalline propylene-based polymer portion and contains only a propylene-ethylene random copolymer, W40 directly contributes to the content, in the whole, of the propylene-ethylene random copolymer portion derived from the fraction 1. The fraction 1 however contains a small amount of components (a component having an extremely low molecular weight and atactic polypropylene) derived from the crystalline propylene-based polymer portion in addition to a component derived from the propylene-ethylene random copolymer. Accordingly, correction in consideration of the crystalline propylene-based polymer portion is required. Thus, by multiplying W₄₀ by A₄₀/B₄₀, the amount derived from the component of the propylene-ethylene random copolymer portion in the fraction 1 is calculated. For example, in the case where the average ethylene content (A₄₀) of the fraction 1 is 30% by mass, and the ethylene content (B₄₀) of the propylene-ethylene random copolymer contained in the fraction 1 is 40% by mass, 30/40=¾ (i.e., 75% by mass) of the fraction 1 is derived from the propylene-ethylene random copolymer, and ¼ is derived from the crystalline propylene-based polymer portion. The multiplying operation by A₄₀/B₄₀ in the first term on the right side means the calculation of the contribution of the propylene-ethylene random copolymer on the % by mass (W₄₀) of the fraction 1. The same applies to the second term on the right side. The contribution of the propylene-ethylene random copolymer is calculated for each fraction and summed, thereby giving the content of the propylene-ethylene random copolymer portion.

(i) As described above, the average ethylene contents corresponding to the fractions 1 and 2 obtained by the CFC measurement are expressed by A₄₀ and A₁₀₀, respectively (units are each % by mass). The average ethylene content will be determined in the manner described later.

(ii) The ethylene content corresponding to the peak position in the differential molecular weight distribution curve of the fraction 1 is defined as B40 (unit: % by mass). Regarding Fraction 2, since all the propylene-ethylene random copolymer portion is considered to be eluted at 40° C., the same definition cannot be used, therefore. In the present invention, accordingly, the ethylene content is substantially defined as B₁₀₀=100. Although the B₄₀ and B₁₀₀ each represent the ethylene content of the propylene-ethylene random copolymer portion contained in each fraction, it is substantially impossible to analytically determine this value. This is because there is no means for completely separating and recovering the propylene homopolymer and propylene-ethylene random copolymer existing as a mixture in the fraction. As a result of investigation using a variety of model samples, it has been found that when the ethylene content corresponding to the peak position of the differential molecular weight distribution curve of the fraction 1 is used as B₄₀, the improving effect of the physical properties of the material can be satisfactorily explained. In addition, judging from two reasons that B₁₀₀ has crystallinity derived from the ethylene chain and the amount of the propylene-ethylene random copolymer contained in these fractions is relatively small compared with the amount of the propylene-ethylene random copolymer portion contained in the fraction 1, B₁₀₀ approximated to 100 rather matches the actual state and hardly causes an error in the calculation. The analysis is therefore carried out on the assumption of B₁₀₀=100.

(iii) Because of the above-described reasons, the ratio (Wc) of the propylene-ethylene random copolymer portion is determined in accordance with the following formula.

$$Wc ( \% by mass ) = {W}_{40} \times {A}_{40} / {B}_{40} + {W}_{100} \times {A}_{100} / 100 \qquad ( II )$$

Described specifically, (W₄₀×A₄₀/B₄₀) which is the first term on the right side of the formula (II) represents the content (% by mass) of the propylene-ethylene random copolymer having no crystallinity, and (W₁₀₀×A₁₀₀/100) which is the second term represents the content (% by mass) of the propylene-ethylene random copolymer portion having crystallinity.

As used herein, B₄₀ and the average ethylene contents A₄₀ and A₁₀₀ of the fractions 1 and 2 obtained by the CFC measurement, are determined as follows.

The ethylene content corresponding to the peak position of the differential molecular weight distribution curve is B₄₀. In addition, the sum of products each of which is a product of the mass ratio at each data point and the ethylene content at each data point, becomes the average ethylene content A₄₀ of the fraction 1, each of the data points being acquired as a data point at the time of measurement. The average ethylene content A₁₀₀ of the fraction 2 is determined in the same manner.

The meaning of setting the three different fractionation temperatures is as follows. In the CFC analysis of the present invention, 40° C. is a temperature condition necessary and sufficient for fractionating only polymers having no crystallinity (for example, the majority of the propylene-ethylene random copolymer, or a component having an extremely low molecular weight and an atactic component in the crystalline propylene-based polymer portion); 100° C. is a temperature necessary and sufficient for eluting only components insoluble at 40° C. but soluble at 100° C. (for example, a component having crystallinity due to the ethylene and/or propylene chain in the propylene-ethylene random copolymer, and the crystalline propylene-based polymer portion); and 140° C. is a temperature necessary and sufficient for eluting only components insoluble at 100° C. but soluble at 140° C. (for example, a component having especially high crystallinity in the crystalline propylene-based polymer portion and a component having an extremely high molecular weight and extremely high ethylene crystallinity in the propylene-ethylene random copolymer) and recovering the whole amount of the propylene-based block copolymer to be provided for the analysis. Since W₁₄₀ does not contain the propylene-ethylene random copolymer component at all or, if any, contains an extremely small amount substantially negligible, W₁₄₀ is excluded from the calculation of the ratio of the propylene-ethylene random copolymer or the ethylene content of the propylene-ethylene random copolymer.

### (2-6) Ethylene Content (Gv) of the Propylene-Ethylene Random Copolymer Portion

The ethylene content of the propylene-ethylene random copolymer portion in the propylene block copolymer of the present invention, can be determined in accordance with the following formula by using the values explained above.

$$Ethylene content ( \% by mass ) of the propylene - ethylene random copolymer portion = ( {W}_{40} \times {A}_{40} + {W}_{100} \times {A}_{100} ) / Wc$$

In the formula, Wc represents the ratio (% by mass) of the propylene-ethylene random copolymer portion determined previously.

### (2-7) Measurement of Intrinsic Viscosity

The intrinsic viscosities [η]p of the crystalline propylene-based polymer portion and propylene-ethylene random copolymer portion in the propylene-based block copolymer of the present invention, are measured at a temperature of 135° C. by an Ubbelohde viscometer while using decalin as a solvent.

After completion of the polymerization of the crystalline propylene-based polymer portion, the intrinsic viscosity [η]p of some of the polymer sampled from a polymerization reactor is measured. Next, the intrinsic viscosity [η]F of the final polymer (F) obtained by the polymerization of the crystalline propylene-based polymer portion and then the polymerization of propylene-ethylene random copolymer, is measured. The [η]c is determined from the below-described relation.

$$[ η ] F = ( 100 - Wc ) / 100 \times [ η ] p + Wc / 100 \times [ η ] c$$

### (3) Evaluation Method of Aggregated Lump Polymers

Aggregated lump polymers were evaluated by the following method, as the index of continuous production stability in the present invention. In particular, the final product was passed through a 5.5-mesh sieve. When 3% by mass (the whole amount of the polymer used for the evaluation is considered as 100% by mass) or more of the polymer remained on the sieve, the result was evaluated as “x”. When less than 3% by mass of the polymer remained on the sieve, the result was evaluated as “∘”.

### (4) Evaluation Methods of Odor and Color

The evaluation of odor was carried out by the following method in the present invention. First, 100 g of the propylene-based polymer was put in a clean glass bottle; the bottle was capped, and the capped glass bottle was heated in an oven at 100° C. for 4 hours; and immediately after the glass bottle was removed from the oven, the bottle was opened up to evaluate the presence of an odor derived from alcohol, sulfur or the like. When such an odor was present, the result was evaluated as “x”. When such an odor was not present, the result was evaluated as “∘”.

The evaluation of color was carried out as follows. A similar sample was used and heated in an oven at 100° C. for 4 hours. Immediately after the sample was removed from the oven, it was visually evaluated whether yellowing or the like occurred in the sample or not. When a change in color was found, the result was evaluated as “x”. When there was no change in color, the result was evaluated as “∘”.

### (5) Measurement of Flexural Modulus

A test piece was formed by use of an injection molding machine (product name: EC100, manufactured by: Shibaura Machine Co., Ltd.) The test piece was conditioned for seven days in a constant temperature room controlled at a temperature of 23° C.±1° C. and a relative humidity of 50%±5%. Using the conditioned test piece, the flexural modulus was obtained in accordance with JIS K7171.

### (6) Evaluation Criteria of Environmental Suitability

In the production of the propylene-based polymer, when the reaction inhibitor derived from the biomass raw material containing impurities at specific concentrations or less, was used as an alternative to a reaction inhibitor derived from a fossil resource, the environmental suitability was evaluated as “∘”. When a reaction inhibitor derived from a fossil resource was used, the environmental suitability was evaluated as “x”. When the production of the propylene-based polymer does not conform to none of them, the environmental suitability was evaluated as “-”.

### (7) Analysis of the Amounts of Impurities in the Reaction Inhibitor

The content of the water was measured by use of a Karl Fischer water content meter in accordance with JIS K8101.

The content of the methanol was measured by gas chromatography using a hydrogen flame ionization detector, in accordance with JAAS001 6.4.

The content of the copper atoms was measured by use of ICP-AES in accordance with JIS K0101 51.2, after the treatment of adding 9 mL of 1.0 M nitric acid per mL of the reaction inhibitor.

The content of the sulfur atoms was measured by the ultraviolet fluorescence method in accordance with JIS K2541-6, after combusting and decomposing the reaction inhibitor in an argon/oxygen atmosphere.

(8) Confirmation of being Biomass-Derived Reaction Inhibitor

The reaction inhibitor (ethanol) used was confirmed to be a biomass-derived reaction inhibitor by the following bio-based content measuring method using oxygen isotope ¹⁸O.

Using a clean microsyringe, 0.2 μL of a measurement sample (ethanol) was directly injected into the pyrolytic furnace of pyrolysis-type elemental analyzer pretreatment system TC/EA (manufactured by THERMO ELECTRON Co., Ltd.) The injected sample was decomposed in the pyrolytic furnace at a temperature of 1400° C. Then, a decomposition gas thus produced was fed into an isotope ratio mass spectrometer (product name: DELTA PLUS XP, manufactured by: Thermo Electoron Co., Ltd.) to measure the oxygen isotope abundance ratio of 160 and 180.

Next, the oxygen isotope abundance ratio of VPDB (Vienna PDB), which is a standard substance of hydrogen isotope D and oxygen isotope ¹⁸O, was measured.

By substituting these oxygen isotope abundance ratio values into the following formula, the oxygen isotope abundance rate parameter δ¹⁸O of the measurement sample (ethanol) was finally obtained.

$${δ}^{18} O = ( the oxygen isotope abundance ratio of the sample / the oxygen isotope abundance ratio of the standard substance - 1 ) \times 1000$$

Using the δ¹⁸O, the value of the oxygen isotope abundance ratio of the sample was evaluated.

When the δ¹⁸O value calculated by the calculation formula is more than zero, the reaction inhibitor used was determined as biomass-derived ethanol. When the δ¹⁸O value was equal to or less than zero, it was determined as a petrochemical-derived ethanol.

### Catalyst Production Example

### (1) Preparation of Solid Catalyst Component for Olefin Polymerization

A 10 L autoclave equipped with a stirrer for preparation of solid components was sufficiently replaced with nitrogen, and 2 L of refined toluene was fed into the autoclave. At room temperature, 200 g of Mg(OEt)₂ and 1 L of TiCl₄ were added. After increasing the temperature of the autoclave to 90° C., 40 ml of di-n-butyl phthalate and 10 ml of diethyl phthalate were fed into the autoclave. Then, the temperature was increased to 110° C. to perform a reaction for 3 hours. A reaction product thus obtained was washed enough with refined toluene. Then, refined toluene was fed to control the whole solution amount to 2 L. At room temperature, 1 L of TiCl₄ was added, and the temperature was increased to 110° C. to perform a reaction for 2 hours. A reaction product thus obtained was washed enough with purified toluene. Then, refined toluene was fed to control the whole solution amount to 2 L. At room temperature, 1 L of TiCl₄ was added, and the temperature was increased to 110° C. to perform a reaction for 2 hours. A reaction product thus obtained was washed enough with purified toluene. In addition, using refined n-heptane, the toluene was substituted by the n-heptane, thereby obtaining a solid component slurry. A part of the slurry was sampled, dried and analyzed. As a result, the Ti content of the solid component was 1.7% by mass. Next, a 20 L autoclave equipped with a stirrer was sufficiently purged with nitrogen, and the solid component slurry in an amount of 100 g (0.036 mol Ti) was fed into the autoclave. Refined n-heptane was fed into the autoclave to control the concentration of the solid component to 25 g/L. Then, 50 ml of SiCl₄ was added, and a reaction was performed at 90° C. for one hour. A reaction product thus obtained was washed enough with refined n-heptane, and the liquid level was controlled to 4 L by feeding refined n-heptane. Next, 25 ml of [CH₂═CH—]₂SiMe₂, 18 ml of (i-Pr)₂Si(OMe)₂, and a diluted solution of triethylaluminum in n-heptane in an amount of 40 g (0.35 mol) in terms of triethylaluminum, were added thereto, and a reaction was performed at 40° C. for two hours. A reaction product thus obtained was washed enough with refined n-heptane.

A part of a slurry thus obtained was sampled, dried and analyzed. As a result, 1.3% by mass of Ti and 7.7% by mass of (i-Pr)₂Si(OMe)₂ were contained in the solid component.

### (2) Preparation of Preliminary Polymerization Catalyst for Olefin Polymerization

Using 100 g (0.025 mol Ti) of the solid component obtained above, preliminary polymerization was carried out by the following process. The refined n-heptane was fed into the above slurry to control the concentration of the solid component to 20 g/L. After cooling the slurry to 10° C., a diluted solution of triethylaluminum in n-heptane, which was in an amount of 15 g (0.132 mol) in terms of triethylaluminum, was added thereto, and 280 g of propylene was supplied over 4 hours. After the propylene supply was completed, the reaction was further continued for 30 minutes. Then, a vapor phase thus formed was sufficiently replaced with nitrogen, and the reaction product was sufficiently washed with refined n-heptane. A slurry thus obtained was drawn from the autoclave and subjected to vacuum drying, thereby obtaining a solid catalyst component. As a result of analyzing the solid catalyst component, it was found that 1.9 g of polypropylene was contained per gram of the solid component, and 0.88% by mass of Ti and 6.8% by mass of (i-Pr)₂Si(OMe)₂ were contained in a part of the solid catalyst component, which was obtained by excluding the polypropylene therefrom.

### Example 1

### (First Polymerization Step)

A stainless-steel autoclave (inner volume 3.0 L) equipped with a stirrer and a temperature controller was heat-dried by vacuumization and then cooled to room temperature. Then, the inside of the autoclave was replaced with propylene gas, and 70.7 mg of triethylaluminum was fed into the autoclave. Then, 9000 mL of hydrogen was fed thereinto, followed by 1000 g of liquefied propylene. After the internal temperature was adjusted to 60° C., the preliminary polymerization catalyst for olefin polymerization was injected by argon so that the solid catalyst component excluding polypropylene was 5.0 mg, thereby initiating the polymerization of the first polypropylene-based polymer. After the lapse of one hour, an unreacted liquefied monomer was purged to stop the polymerization. A part of the polymer thus formed was analyzed by MFR. As a result, the polymer was a crystalline polypropylene polymer having a melt flow rate of 107 g/10 min.

After sampling a part of the polymer produced in the first step, 10 mg of bioethanol (biomass-derived ethanol purchased from FUJIFILM Wako Pure Chemical Corporation) was fed into the reactor. Then, they were stirred for 5 minutes by use of a stirrer to bring the bioethanol into contact with the first propylene-based polymer produced in the first step. In this manner, the biomass-derived reaction inhibitor was added at the midpoint of the first and second steps.

The bioethanol used contained the following impurities: 1688 ppm by mass of water, 428 ppm by mass of methanol, 2.4 ppm by mass of sulfur, and 54 ppb by mass of copper.

### (Second Polymerization Step)

Using an autoclave (inner volume 20 L) equipped with a stirrer and a temperature controller, which is different from the polymerization reactor used in the first process, a gas to be used for polymerization in the second step was adjusted. The adjusting temperature was 95° C. The composition of the mixed gas was as follows: hydrogen 0.54 mol %, propylene 62.7 mol %, ethylene 36.3 mol % and nitrogen 0.50 mol %.

After the contact with the reaction inhibitor was completed, the temperature of the 3.0 L autoclave was increased to 70° C., and the mixed gas was supplied thereto until the total pressure of the reactor became 1.0 MPaG, thereby initiating the polymerization of the second step. The polymerization reaction was carried out for one hour while keeping the polymerization temperature at 70° C. and the reaction pressure at 1.0 MPaG; moreover, the polymerization reaction was stopped by purging an unreacted residual monomer, thereby obtaining a propylene-based polymer-1.

The obtained polymer was dried under reduced pressure at 90° C. for one hour. Then, several analyses of the dried polymer were carried out, and the following results were obtained: MFR=7.7 g/10 min; the ratio (Wc) of the propylene-based copolymer portion produced in the second step was 32.2% by mass; the ethylene content (Gv) in the propylene-ethylene copolymer was 41% by mass; and the intrinsic viscosity (η) of the propylene-ethylene copolymer was 4.0 dL/g. As a result of the sensory test, the evaluation result of the odor was excellent, and the visual evaluation of the powder color resulted in “excellent appearance”. The measured value of the flexural modulus was 860 MPa.

### Example 2

A propylene-based polymer-2 was obtained by changing the production method of Example 1 as follows.

The amount of the reaction inhibitor supplied at the midpoint of the first and second steps, was changed to 20 mg.

The composition of the mixed gas used in the second step was changed to the following composition: propylene 54.5 mol %, ethylene 36.3 mol %, hydrogen 0.54 mol % and nitrogen 8.7 mol %.

The propylene-based polymer-2 was produced in the same manner as Example 1, except for the above changes.

As a result of analyzing the obtained propylene-based polymer-2, the following results were obtained: MFR=13 g/10 min; the ratio (Wc) of the propylene-based copolymer portion produced in the second step was 25.5% by mass; the ethylene content (Gv) in the propylene-ethylene copolymer was 41% by mass; and the intrinsic viscosity (η) of the propylene-ethylene copolymer was 4.0 dL/g. As a result of the sensory test, the evaluation result of the odor was excellent, and the visual evaluation of the powder color also resulted in “excellent appearance”. The measured value of the flexural modulus was 1000 MPa.

### Example 3

A propylene-based polymer-3 was obtained by changing the production method of Example 1 as follows.

The amount of the reaction inhibitor supplied at the midpoint of the first and second steps, was changed to 40 mg.

The composition of the mixed gas used in the second step was changed to the following composition: propylene 56.0 mol %, ethylene 34.0 mol %, hydrogen 0.60 mol % and nitrogen 9.4 mol %.

The propylene-based polymer-3 was produced in the same manner as Example 1, except for the above changes.

As a result of analyzing the obtained propylene-based polymer-3, the following results were obtained: MFR=88 g/10 min; the ratio (Wc) of the propylene-based copolymer portion produced in the second step was 3.0% by mass; the ethylene content (Gv) in the propylene-ethylene copolymer was 40% by mass; and the intrinsic viscosity (η) of the propylene-ethylene copolymer was 3.5 dL/g. As a result of the sensory test, the evaluation result of the odor was excellent, and the visual evaluation of the powder color also resulted in “excellent appearance”. The measured value of the flexural modulus was 1900 MPa.

### Example 4

A propylene-based polymer-4 was obtained by changing the production method of Example 1 as follows.

The amount of the reaction inhibitor supplied at the midpoint of the first and second steps, was changed to 20 mg.

The composition of the mixed gas used in the second step was changed to the following composition: propylene 54.5 mol %, ethylene 36.3 mol %, hydrogen 0.54 mol % and nitrogen 8.7 mol %. Also, using dimethyl sulfide (purchased from FUJIFILM Wako Pure Chemical Corporation), a super dehydrated hexane solution with a concentration of 8.4×10⁻⁵ mg/mL was adjusted and added at the same feed timing as the reaction inhibitor.

The propylene-based polymer-4 was produced in the same manner as Example 1, except for the above changes.

As a result of analyzing the obtained propylene-based polymer-4, the following results were obtained: MFR=14 g/10 min; the ratio (Wc) of the propylene-based copolymer portion produced in the second step was 24.5% by mass; the ethylene content (Gv) in the propylene-ethylene copolymer was 43% by mass; and the intrinsic viscosity (η) of the propylene-ethylene copolymer was 4.0 dL/g. As a result of the sensory test, the evaluation result of the odor was excellent, and the visual evaluation of the powder color also resulted in “excellent appearance”. The measured value of the flexural modulus was 950 MPa.

### Example 5

A propylene-based polymer-5 was obtained by changing the production method of Example 1 as follows.

The amount of the reaction inhibitor supplied at the midpoint of the first and second steps, was changed to 20 mg.

The composition of the mixed gas used in the second step was changed to the following composition: propylene 54.5 mol %, ethylene 36.3 mol %, hydrogen 0.54 mol % and nitrogen 8.7 mol %. Also, using copper acetate (purchased from FUJIFILM Wako Pure Chemical Corporation), a super dehydrated hexane solution with a concentration of 1.0×10⁻⁷ mg/mL was adjusted and added at the same feed timing as the reaction inhibitor.

The propylene-based polymer-5 was produced in the same manner as Example 1, except for the above changes.

As a result of analyzing the obtained propylene-based polymer-2, the following results were obtained: MFR=14 g/10 min; the ratio (Wc) of the propylene-based copolymer portion produced in the second step was 24.9% by mass; the ethylene content (Gv) in the propylene-ethylene copolymer was 42% by mass; and the intrinsic viscosity (η) of the propylene-ethylene copolymer was 4.0 dL/g. As a result of the sensory test, the evaluation result of the odor was excellent, and the visual evaluation of the powder color also resulted in “excellent appearance”. The measured value of the flexural modulus was 930 MPa.

### Comparative Example 1

A propylene-based polymer-C1 was obtained by changing the production method of Example 1 as follows.

The reaction inhibitor supplied at the midpoint of the first and second steps, was changed to petrochemical-derived ethanol (grade: super dehydrated, purchased from: FUJIFILM Wako Pure Chemical Corporation). The petrochemical-derived ethanol contained the following impurities: 10 ppm by mass or less of water and 20 ppm by mass or less of methanol. Sulfur and copper atoms were not detected therefrom.

The propylene-based polymer-C1 was produced in the same manner as Example 1, except for the above changes.

As a result of analyzing the obtained propylene-based polymer-C1, the following results were obtained: MFR=9.2 g/10 min; the ratio (Wc) of the propylene-based copolymer portion produced in the second step was 30% by mass; the ethylene content (Gv) in the propylene-ethylene copolymer was 40% by mass; and the intrinsic viscosity (η) of the propylene-ethylene copolymer was 4.0 dL/g. As a result of the sensory test, the evaluation result of the odor was excellent, and the visual evaluation of the powder color also resulted in “excellent appearance”. The measured value of the flexural modulus was 850 MPa.

### Comparative Example 2

A propylene-based polymer-C2 was obtained by changing the production method of Comparative Example 1 as follows.

The amount of the reaction inhibitor supplied at the midpoint of the first and second steps, was changed to 20 mg.

The propylene-based polymer-C2 was produced in the same manner as Example 1, except for the above change.

As a result of analyzing the obtained propylene-based polymer-C2, the following results were obtained: MFR=21 g/10 min; the ratio (Wc) of the propylene-based copolymer portion produced in the second step was 20% by mass; the ethylene content (Gv) in the propylene-ethylene copolymer was 40% by mass; and the intrinsic viscosity (η) of the propylene-ethylene copolymer was 4.0 dL/g. As a result of the sensory test, the evaluation result of the odor was excellent, and the visual evaluation of the powder color also resulted in “excellent appearance”. The measured value of the flexural modulus was 1220 MPa.

### Comparative Example 3

A propylene-based polymer-C3 was obtained by changing the production method of Comparative Example 1 as follows.

The amount of the reaction inhibitor supplied at the midpoint of the first and second steps, was changed to 40 mg.

The propylene-based polymer-C3 was produced in the same manner as Example 1, except for the above change.

As a result of analyzing the obtained propylene-based polymer-C3, the following results were obtained: MFR=91 g/10 min; the ratio (Wc) of the propylene-based copolymer portion produced in the second step was 2.0% by mass; the ethylene content (Gv) in the propylene-ethylene copolymer was 40% by mass; and the intrinsic viscosity (η) of the propylene-ethylene copolymer was 4.0 dL/g. As a result of the sensory test, the evaluation result of the odor was excellent, and the visual evaluation of the powder color also resulted in “excellent appearance”. The measured value of the flexural modulus was 1900 MPa.

### Comparative Example 4

A propylene-based polymer-C4 was obtained by changing the production method of Comparative Example 1 as follows.

Any reaction inhibitor was not used.

The propylene-based polymer-C4 was produced in the same manner as Example 1, except for the above change.

As a result of analyzing the obtained propylene-based polymer-C4, the following results were obtained: MFR=2.1 g/10 min; the ratio (Wc) of the propylene-based copolymer portion produced in the second step was 48% by mass; the ethylene content (Gv) in the propylene-ethylene copolymer was 40% by mass; and the intrinsic viscosity (η) of the propylene-ethylene copolymer was 4.0 dL/g. As a result of the sensory test, the evaluation result of the odor was excellent, and the visual evaluation of the powder color also resulted in “excellent appearance”. As a result of passing the propylene-based polymer-C4 through a sieve having openings 3350 μm in size, the formation of 5% by mass of aggregated lump polymers were found. The measured value of the flexural modulus was 510 MPa.

### Comparative Example 5

A propylene-based polymer-C5 was obtained by changing the production method of Comparative Example 1 as follows.

The amount of the reaction inhibitor supplied at the midpoint of the first and second steps, was changed to 20 mg.

The propylene-based polymer-C5 was produced in the same manner as Example 1, except for the above change.

As a result of analyzing the obtained propylene-based polymer-C5, the following results were obtained: MFR=14 g/10 min; the ratio (Wc) of the propylene-based copolymer portion produced in the second step was 24.9% by mass; the ethylene content (Gv) in the propylene-ethylene copolymer was 42% by mass; and the intrinsic viscosity (η) of the propylene-ethylene copolymer was 4.0 dL/g. As a result of the sensory test, the evaluation result of the odor was poor, and the visual evaluation of the powder color also resulted in “poor appearance”. The measured value of the flexural modulus was 950 MPa.

### Comparative Example 6

A propylene-based polymer-C6 was obtained by changing the production method of Comparative Example 1 as follows.

The amount of the reaction inhibitor supplied at the midpoint of the first and second steps, was changed to 20 mg.

The propylene-based polymer-C6 was produced in the same manner as Example 1, except for the above change.

As a result of analyzing the obtained propylene-based polymer-C6, the following results were obtained: MFR=9.2 g/10 min; the ratio (Wc) of the propylene-based copolymer portion produced in the second step was 30% by mass; the ethylene content (Gv) in the propylene-ethylene copolymer was 40% by mass; and the intrinsic viscosity (η) of the propylene-ethylene copolymer was 4.0 dL/g. As a result of the sensory test, the evaluation result of the odor was poor, and the visual evaluation of the powder color resulted in “excellent appearance”. The measured value of the flexural modulus was 710 MPa.

The polymerization results and the evaluation results are shown in Tables 1 and 2.

TABLE 1

| | | | Example 1 | Example 2 | Example 3 | Example 4 | Example 5 |
|---|---|---|---|---|---|---|---|
| Catalyst | Naked catalyst | mg | 5.0 | 5.0 | 5.0 | 5.0 | 5.0 |
| | amount | | | | | | |
| | Ti content | wt. % | 0.88 | 0.88 | 0.88 | 0.88 | 0.88 |
| Organoaluminum | Type | | TEA | TEA | TEA | TEA | TEA |
| | | mg | 70.7 | 70.7 | 70.7 | 70.7 | 70.7 |
| First | Inner volume | L | 3 | 3 | 3 | 3 | 3 |
| step | Polymerization | | Bulk | Bulk | Bulk | Bulk | Bulk |
| | type | | | | | | |
| | Polymerization | ° C. | 60 | 60 | 60 | 60 | 60 |
| | temperature | | | | | | |
| | Polymerization | hr | 1 | 1 | 1 | 1 | 1 |
| | time | | | | | | |
| | propylene | g | 1000 | 1000 | 1000 | 1000 | 1000 |
| | amount | | | | | | |
| | H2 charge | NL | 9000 | 9000 | 9000 | 9000 | 9000 |
| | MFR | g/10 min | 107 | 107 | 107 | 107 | 107 |
| | Polymer yield | g | 270 | 270 | 270 | 270 | 270 |
| | Polymer | g/g | 54000 | 54000 | 54000 | 54000 | 54000 |
| | yield/Catalyst | | | | | | |
| | amount | | | | | | |
| Reaction | Type 1 | | Bioethanol | Bioethanol | Bioethanol | Bioethanol | Bioethanol |
| inhibitor | Type 2 | | — | — | — | Dimethyl | Copper |
| | | | | | | sulfide | acetate |
| | Ethanol | mg | 10 | 20 | 40 | 20 | 20 |
| | amount | | | | | | |
| | EtOH/Catalyst | g/g | 2.0 | 4.0 | 8.0 | 4.0 | 4.0 |
| | Water content | ppm | 1688 | 1688 | 1688 | 1615 | 1674 |
| | Methanol | ppm | 428 | 428 | 428 | 409 | 425 |
| | content | | | | | | |
| | S content | ppm | 2.4 | 2.4 | 2.4 | 4.7 | 2.4 |
| | Cu content | ppb | 54 | 54 | 54 | 51 | 95 |
| Second | Inner volume | L | 3 | 3 | 3 | 3 | 3 |
| step | Polymerization | | Gas | Gas | Gas | Gas | Gas |
| | type | | | | | | |
| | Polymerization | ° C. | 70 | 70 | 70 | 70 | 70 |
| | temperature | | | | | | |
| | Total pressure | MPaG | 1.0 | 1.0 | 1.0 | 1.0 | 1.0 |
| | PPY | mol % | 62.7 | 54.5 | 56.0 | 54.5 | 54.5 |
| | concentration | | | | | | |
| | ETY | mol % | 36.3 | 36.3 | 34.0 | 36.3 | 36.3 |
| | concentration | | | | | | |
| | H2 | mol % | 0.54 | 0.54 | 0.60 | 0.54 | 0.54 |
| | concentration | | | | | | |
| | N2 | mol % | 0.50 | 8.70 | 9.40 | 8.70 | 8.70 |
| | Total | mol % | 100.0 | 100.0 | 100.0 | 100.0 | 100.0 |
| | ETY/PPY | m.r. | 0.58 | 0.67 | 0.61 | 0.67 | 0.67 |
| | H2/Monomer | m.r. | 0.01 | 0.01 | 0.01 | 0.01 | 0.01 |
| | Reaction time | hr | 1 | 1 | 1 | 1 | 1 |
| | Wc | wt. % | 32.2 | 25.5 | 3.0 | 24.5 | 24.9 |
| | Gv | wt. % | 41 | 41 | 40 | 43 | 42 |
| | ηcxs | dL/g | 4.0 | 4.0 | 3.5 | 4.0 | 4.0 |
| | MFR | g/10 min | 7.7 | 13 | 88 | 14 | 14 |
| Product | Formation of | — | ∘ | ∘ | ∘ | ∘ | ∘ |
| analysis | aggregated | | | | | | |
| | lump polymers | | | | | | |
| | Color | — | ∘ | ∘ | ∘ | ∘ | ∘ |
| | Odor | — | ∘ | ∘ | ∘ | ∘ | ∘ |
| | Flexural | MPa | 860 | 1000 | 1900 | 950 | 930 |
| | modulus | | | | | | |
| Environmental | | | ∘ | ∘ | ∘ | ∘ | ∘ |
| suitability | | | | | | | |

TABLE 2

| | | | Comparative | Comparative | Comparative | Comparative | Comparative | Comparative |
|---|---|---|---|---|---|---|---|---|
| | | | Example 1 | Example 2 | Example 3 | Example 4 | Example 5 | Example 6 |
| Catalyst | Naked catalyst | mg | 5.0 | 5.0 | 5.0 | 5.0 | 5.0 | 5.0 |
| | amount | | | | | | | |
| | Ti content | wt. % | 0.88 | 0.88 | 0.88 | 0.88 | 0.88 | 0.88 |
| Organoaluminum | Type | | TEA | TEA | TEA | TEA | TEA | TEA |
| | | mg | 70.7 | 70.7 | 70.7 | 70.7 | 70.7 | 70.7 |
| First | Inner volume | L | 3 | 3 | 3 | 3 | 3 | 3 |
| step | Polymerization | | Bulk | Bulk | Bulk | Bulk | Bulk | Bulk |
| | type | | | | | | | |
| | Polymerization | ° C. | 60 | 60 | 60 | 60 | 60 | 60 |
| | temperature | | | | | | | |
| | Polymerization | hr | 1 | 1 | 1 | 1 | 1 | 1 |
| | time | | | | | | | |
| | propylene | g | 1000 | 1000 | 1000 | 1000 | 1000 | 1000 |
| | amount | | | | | | | |
| | H2 charge | NL | 9000 | 9000 | 9000 | 9000 | 9000 | 9000 |
| | MFR | g/10 min | 107 | 107 | 107 | 107 | 107 | 107 |
| | Polymer yield | g | 270 | 270 | 270 | 270 | 270 | 270 |
| | Polymer | g/g | 54000 | 54000 | 54000 | 54000 | 54000 | 54000 |
| | yield/Catalyst | | | | | | | |
| | amount | | | | | | | |
| Reaction | Type 1 | | Super | Super | Super | None | Super | Super |
| inhibitor | | | dehydrated | dehydrated | dehydrated | | dehydrated | dehydrated |
| | | | ethanol | ethanol | ethanol | | ethanol | ethanol |
| | Type 2 | | — | — | — | — | Dimethyl | Copper |
| | | | | | | | sulfide | acetate |
| | Ethanol amount | mg | 10 | 20 | 40 | 0 | 20 | 20 |
| | EtOH/Catalyst | g/g | 2.0 | 4.0 | 8.0 | 0.0 | 4.0 | 4.0 |
| | Water | ppm | 10 ppm | 10 ppm | 10 ppm | 10 ppm | 10 ppm | 10 ppm |
| | content | | or less | or less | or less | or less | or less | or less |
| | Methanol | ppm | 20 ppm | 20 ppm | 20 ppm | 20 ppm | 20 ppm | 20 ppm |
| | content | | or less | or less | or less | or less | or less | or less |
| | S content | ppm | Below the | Below the | Below the | Below the | 10 | Below the |
| | | | limit of | limit of | limit of | limit of | | limit of |
| | | | quantification | quantification | quantification | quantification | | quantification |
| | Cu content | ppb | Not detected | Not detected | Not detected | Not detected | Not detected | 775 |
| Second | Inner volume | L | 3 | 3 | 3 | 3 | 3 | 3 |
| step | Polymerization | | Gas | Gas | Gas | Gas | Gas | Gas |
| | type | | | | | | | |
| | Polymerization | ° C. | 70 | 70 | 70 | 70 | 70 | 70 |
| | temperature | | | | | | | |
| | Total pressure | MPaG | 1.0 | 1.0 | 1.0 | 1.0 | 1.0 | 1.0 |
| | PPY | mol % | 62.7 | 62.7 | 62.7 | 62.7 | 62.7 | 62.7 |
| | concentration | | | | | | | |
| | ETY | mol % | 36.3 | 36.3 | 36.3 | 36.3 | 36.3 | 36.3 |
| | concentration | | | | | | | |
| | H2 | mol % | 0.54 | 0.54 | 0.54 | 0.54 | 0.54 | 0.54 |
| | concentration | | | | | | | |
| | N2 | mol % | 0.50 | 0.50 | 0.50 | 0.50 | 0.50 | 0.50 |
| | Total | mol % | 100.0 | 100.0 | 100.0 | 100.0 | 100.0 | 100.0 |
| | ETY/PPY | m.r. | 0.58 | 0.58 | 0.58 | 0.58 | 0.58 | 0.58 |
| | H2/Monomer | m.r. | 0.01 | 0.01 | 0.01 | 0.01 | 0.01 | 0.01 |
| | Reaction time | hr | 1 | 1 | 1 | 1 | 1 | 1 |
| | Wc | wt. % | 30 | 20 | 2 | 48 | 24.9 | 30 |
| | Gv | wt. % | 40 | 40 | 40 | 40 | 42 | 40 |
| | ηcxs | dL/g | 4.0 | 4.0 | 4.0 | 4.0 | 4.0 | 4.0 |
| | MFR | g/10 min | 9.2 | 21 | 91 | 2.1 | 14.0 | 9.2 |
| Product | Formation of | — | ∘ | ∘ | ∘ | x | ∘ | ∘ |
| analysis | aggregated | | | | | | | |
| | lump polymers | | | | | | | |
| | Color | — | ∘ | ∘ | ∘ | ∘ | x | ∘ |
| | Odor | — | ∘ | ∘ | ∘ | ∘ | x | x |
| | Flexural | MPa | 850 | 1220 | 1900 | 510 | 950 | 710 |
| | modulus | | | | | | | |
| Environmental | | | x | x | x | — | x | x |
| suitability | | | | | | | | |

As a result of comparing Examples 1, 2 and 3 to Comparative Examples 1, 2 and 3, respectively, it was revealed that compared to Comparative Example 4 in which any reaction inhibitor was not used, Examples 1 to 3 can offer, in spite of their use of the reaction inhibitor containing a certain amount of biomass-derived impurities, comparable production to Comparative Examples 1 to 3 in each of which the fossil fuel-derived reaction inhibitor was used.

From the results of Examples 1 to 5 and Comparative Examples 1 to 6, it was revealed that even when bioethanol containing a certain amount of impurities is used as the reaction inhibitor, it can be used without causing a remarkable decrease in productivity and a long-term unstable operation owing to the formation of lump polymers, and it is good to the environment since it is biomass-derived ethanol. Also, it was revealed that when the water content of the reaction inhibitor is more than the predetermined amount, production problems (such as an equipment failure induced by the water deposited in a production equipment) can occur; however, when the water content is less than the predetermined amount, the reaction inhibitor can be used without any production problem.

1. A method for producing a propylene-based polymer comprising:
producing a first propylene-based polymer using one or two or more polymerization reactors and in a presence of an olefin polymerization catalyst, a propylene homopolymer or a copolymer of propylene and at least one kind of monomer selected from the group consisting of α-olefins containing 2 to 10 carbon atoms other than propylene;
producing a second propylene-based polymer using one or two or more polymerization reactors and in a presence of the first propylene-based polymer, a propylene homopolymer or a copolymer of propylene and at least one kind of monomer selected from the group consisting of α-olefins containing 2 to 10 carbon atoms other than propylene; and
wherein at least one selected from the group consisting of producing the first propylene-based polymer, producing the second propylene-based polymer, and a midpoint of the first and second propylene-based polymer productions includes addition of a biomass-derived reaction inhibitor containing 5 ppm by mass to 2000 ppm by mass of water.

2. The method for producing the propylene-based polymer according to claim 1, wherein the reaction inhibitor further contains 0.1 ppm by mass to 1000 ppm by mass of methanol.

3. The method for producing the propylene-based polymer according to claim 1, wherein the reaction inhibitor further contains 0.1 ppm by mass to 5 ppm by mass of sulfur atoms and 0.1 ppb by mass to 100 ppb by mass of copper atoms.

4. The method for producing the propylene-based polymer according to claim 1, wherein the reaction inhibitor is biomass-derived ethanol.

5. The method for producing the propylene-based polymer according to claim 1, wherein the olefin polymerization catalyst contains a solid catalyst component (A) containing the following (A1), (A2) and (A3) and optionally containing the following (A4), and the following component (B):
(A1) a solid component containing magnesium, titanium, a halogen and an electron donating compound serving as an internal donor
(A2) an organoaluminum compound
(A3) an organosilicon compound other than a vinylsilane compound
(A4) a vinylsilane compound
(B) an organoaluminum compound.

6. The method for producing the propylene-based polymer according to claim 1, wherein the second propylene-based polymer is a copolymer of propylene and at least one kind of monomer selected from the group consisting of α-olefins containing 2 to 10 carbon atoms other than propylene, and a content of the at least one kind of monomer selected from the group consisting of α-olefins containing 2 to 10 carbon atoms other than propylene, is in a range of from 20% by mass to 80% by mass.

7. The method for producing the propylene-based polymer according to claim 5, wherein an amount of the added reaction inhibitor is from 0.01 g to 30 g with respect to 1 g of a total amount of the solid catalyst component (A).
